(19) United States
(12) Patent Application Publication
(10) Pub. No.: US 2024/0199632 A1
(43) Pub. Date: Jun. 20, 2024

(54) BRIARANE COMPOUNDS AND USE THEIR USE

(71) Applicants: National Health Research Institutes, Miaoli County (TW); National Sun Yat-Sen University, Kaohsiung (TW); National Museum of Marine Biology and Aquarium, Pingtung County (TW)

(72) Inventors: Lun Kelvin Tsou, Miaoli County (TW); Guang-Hao Niu, Miaoli County (TW); Hsing-Pang Hsieh, Miaoli County (TW); Mingzi Zhang, Miaoli County (TW); Zhi-Hong Wen, Kaohsiung (TW); Ping-Jyun Sung, Pingtung County (TW)

(21) Appl. No.: 18/517,497

(22) Filed: Nov. 22, 2023

Related U.S. Application Data
(60) Provisional application No. 63/428,303, filed on Nov. 28, 2022.

(51) Int. Cl. C07D 493/04; C07D 307/935; C07D 413/04

(52) U.S. Cl. CPC C07D 493/04; C07D 307/935; C07D 413/04

(57) ABSTRACT

Disclosed are compounds compound of Formula (I):

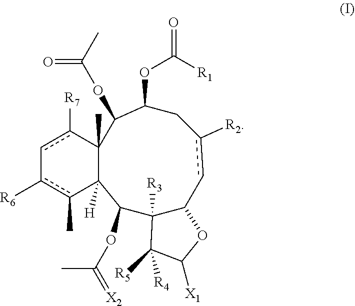

Each variable is defined herein. Also included are pharmaceutical compositions containing such a compound and a method of treating an inflammatory condition.

### CROSS-REFERENCE TO RELATED APPLICATION

This application claims the benefit of priority to U.S. Provisional Application No. 63/428,303 filed on Nov. 28, 2022, the entire content of is incorporated by reference.

### STATEMENT REGARDING SEQUENCE LISTING

The Sequence Listing associated with this application is provided in xml format in lieu of a paper copy and is hereby incorporated by reference into the specification. The name of the xml file containing the Sequence Listing is NHRI-057-101 SEQ LIST.xml. The xml file is 3.47 KB, was created on Dec. 18, 2023, and is being submitted electronically via Patent Center.

### BACKGROUND

Inflammatory reaction serves as a fundamental protective mechanism against infection caused by outside invaders such as viruses or bacteria. It involves various immune cells including macrophages, neutrophils, and lymphocytes. Many of these immune cells express inducible nitric oxide synthase (“iNOS”) and cyclooxygenase (“COX-2”), both of which are pro-inflammatory mediators activating inflammatory responses. Inhibiting iNOS and COX-2 provides treatment of inflammation in patients in need.

Chronic inflammation is associated with many autoimmune diseases such as arthritis, irritable bowel syndrome, and neurodegenerative disorders. In these diseases, the immune system triggers inflammation to attack normal tissues as if they are infected. Although numerous corticosteroids and non-steroidal anti-inflammatory drugs have been developed for treating inflammatory conditions, they often exhibit undesired side effects and drug resistance.

Natural products harbor a rich repertoire for anti-inflammatory agents. Notably, briarane-type diterpenoids found in marine gorgonians have emerged as a new class of anti-inflammatory agents. Of interest, excavatolide B (“ExcB”), obtained from Formosan gorgonian Briareum excavatum, has shown significantly inhibiting the mRNA expression of inducible nitric oxide synthase (iNOS) and cyclooxygenase-2 (COX-2) in lipopolysaccharide (LPS)-challenged murine macrophages. See Berrue et al., Nat. Prod. Rep. 2009, 26, 681-710; and Lin et al., Mar Drugs 2015, 13, 2559-79.

ExcB has not been approved for use as an anti-inflammation drug due to low potency. Further, ExcB has a poor solubility in water and thus low bioavailability, a major challenge for drug development. Currently, it is still not clear what its direct target is, making it difficult to develop potent ExcB derivatives.

There is a need to develop a potent and safe anti-inflammation medicine from ExcB analogs.

### SUMMARY

The present invention is based on an unexpected discovery that certain briarane compounds show high anti-inflammatory potency.

In one aspect, this invention relates to compounds of Formula (I):

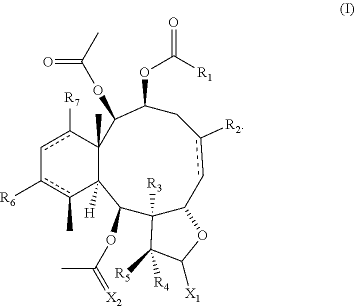

In this formula, R₁ is C₁-C₆ alkyl; R₂ is C₁-C₆ alkyl, formyl, carboxyl, —CH═N—ORₐ, —CH═NRₐ, or —C(O)NH—O—Rₐ, in which Rₐ is H, C₁-C₆ alkyl, or C₁-C₆ (e.g., 3- to 10-membered, 4- to 8-membered, and 5- to 6-membered) heterocycloalkyl; R₃ is H, hydroxy, or combined with R₄, and R₄ is H or C₁-C₆ alkyl; when R₃ and R₄ are combined, they together with the two carbon atoms attached thereto form an epoxide ring; R₅ is N₃, methyl, or combined with X₂, and X₂ is O or N, when R₅ and X₂ are combined, they are N and, together with the carbon atoms attached thereto, form a heterocycloalkyl ring; R₆ is H, halo, oxo, hydroxyl, ═NR_{b}, ═N—NH—C(O)R_{c}, —OC(O)R_{d}, or —O—S(O)₂Rₑ, in which R_{b} is hydroxyl, C₁-C₆ alkoxy, or C₁-C₁₀ heterocycloalkyl, R_{c} is C₁-C₆ alkyl, C₃-C₆ cycloalkyl, or phenyl, R_{d} is C₃-C₆ cycloalkyl, C₁-C₁₀ aminoalkyl, C₃-C₁₀ alkynylalkyl, C₁-C₁₀ heterocyclylalkyl, or phenyl, and Rₑ is amino or C₁-C₆ alkyl; R₇ is H or acetyloxy; X₁ is hydroxyl or oxo; X₂ is O or N, when X₂ is N, it is combined with R₅; and each of [symbol] is a single bond or a double bond.

Alkyl is unsubstituted or substituted with one or more groups selected from hydroxyl, amino, aryl, heteroaryl, C₂-C₆ alkenyl, C₂-C₆ alkynyl, C₁-C₆ carboxylate, and C₁-C₆ (e.g., 3- to 10-membered, 4- to 8-membered, and 5- to 6-membered) heterocycloalkyl.

Each of amino, aminoalkyl, alkoxy, cycloalkyl, alkynylalkyl, heterocyclylalkyl, heterocycloalkyl, heteroaryl, and phenyl, is unsubstituted or substituted with one or more groups selected from hydroxyl, C₁-C₆ alkyl, C₂-C₆ alkenyl, C₂-C₆ alkynyl, C₁-C₆ carboxylate, C₁-C₆ (e.g., 3- to 10-membered, 4- to 8-membered, and 5- to 6-membered) heterocycloalkyl, aryl, and heteroaryl.

The substituents and their subsequent occurrences can be further substituted with the chemical groups described above.

In another embodiment, in Formula (I) above, R₁ is C₁-C₆ alkyl; R₂ is C₁-C₆ alkyl, formyl, carboxyl, —CH═N—ORₐ, —CH═NRₐ, or —C(O)NH—O—Rₐ, in which Rₐ is H, C₁-C₆ alkyl, C₂-C₆ alkynyl, or C₁-C₆ heterocycloalkyl; R₃ is H, hydroxy, or combined with R₄, and R₄ is H or C₁-C₆ alkyl; when R₃ and R₄ are combined, they together with the two carbon atoms attached thereto form an epoxide ring; R₅ is N₃, methyl, or combined with X₂, and when R₅ and X₂ are combined, they are N and, together with the carbon atoms attached thereto, form a heterocycloalkyl ring; R₆ is H, halo, oxo, hydroxyl, ═NR_{b}, ═N—NH—C(O)R_{c}, —OC(O)R_{d}, or —O—S(O)₂Rₑ, in which R_{b} is hydroxyl, C₁-C₆ alkoxy, or C₁-C₁₀ heterocycloalkyl, R_{c} is C₁-C₆ alkyl, C₃-C₆ cycloalkyl, 5 or 6-membered heterocycloalkyl, phenyl, or 5- or 6-membered heteroaryl, R_{d} is C₃-C₆ cycloalkyl, C₂-C₁₀ alkenyl, C₁-C₁₀ aminoalkyl, C₃-C₁₀ alkynylalkyl, C₁-C₁₀ heterocyclylalkyl, or phenyl, and Rₑ is amino or C₁-C₆ alkyl; R₇ is H or acetyloxy; X₁ is hydroxyl or oxo; X₂ is O or N, when X₂ is N, it is combined with R₅; each of [symbol] is a single bond or a double bond; alkyl is unsubstituted or substituted with one or more groups selected from hydroxyl, amino, CN, aryl, heteroaryl, C₂-C₆ alkenyl, C₂-C₆ alkynyl, C₁-C₆ carboxylate, and C₁-C₆ heterocycloalkyl; and each of amino, aminoalkyl, alkenyl, alkoxy, cycloalkyl, alkynylalkyl, heterocyclylalkyl, heterocycloalkyl, heteroaryl, and phenyl, is unsubstituted or substituted with one or more groups selected from hydroxyl, C₁-C₆ alkyl, C₁-C₆ alkenyl, C₁-C₆ alkynyl, C₁-C₆ carboxylate, C₁-C₆ heterocycloalkyl, aryl, heteroaryl, and aryl-amino-alkyl-aminocarbonyl.

Preferably, the above-described compounds have one or more of the following features:
(i) R₂ is formyl, carboxyl, —CH═N—ORₐ, —CH═NRₐ, or —C(O)NH—O—Rₐ; and R₆ is OH;
(ii) each of X₁ and R₆ is OH;
(iii) R₆ is oxo, ═NR_{b}, ═N—NH—C(O)R_{c}, —OC(O)R_{d}, or —O—S(O)₂Rₑ;
(iv) R₁ is methyl, ethyl, n-propyl, or iso-propyl;
(v) each of R₃ and R₄, independently, is H or OH, or R₃ and R₄ are combined and, together with the carbon atoms they bond to, form an epoxide ring;
(vi) R₅ is H, OH, N₃, or methyl; and
(vii) R₇ is acetyloxy.

Subsets of the compounds of Formula (I) are represented by Formulas (I-A), (I-A1), (I-B), (I-C), (I-D), (I-E), (I-F), and (I-G):

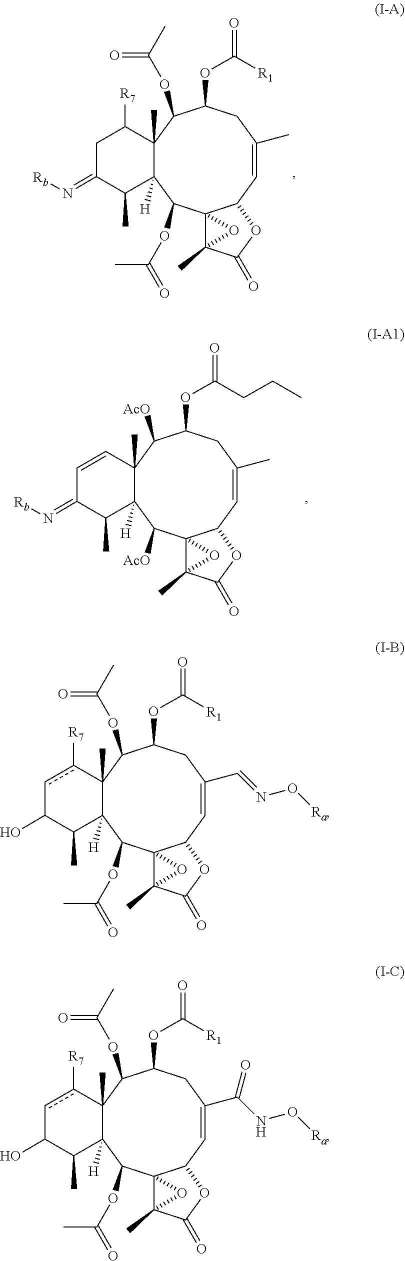

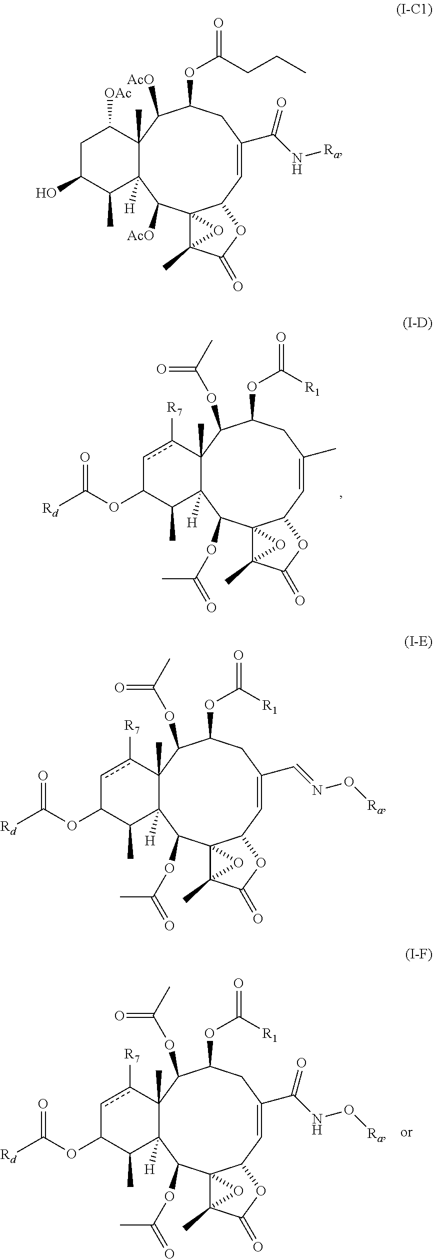

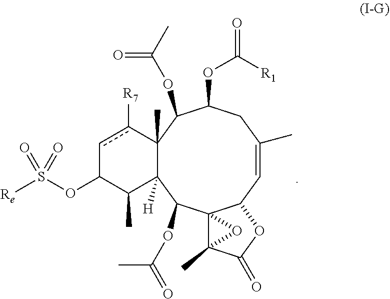

In Formula (I-A) or (I-A1) above, preferably, R_{b} is hydroxyl, C₁-C₆ alkoxy, 5- or 6-membered heterocycloalkyl, or —NH—C(O)R_{c}, R_{c} being C₁-C₆ alkyl, C₃-C₆ cycloalkyl, phenyl, or heteroaryl; more preferably, R₁ is propyl, R₇ is acetyloxy, and R_{b} is hydroxyl, methoxy, CH≡CCH₂O—, PhC(O)NH—, CH₃C(O)NH—, cyclopropanecarbox-amido, N-methylpiperazin-1-yl, or morpholino. Ph is phenyl.

In Formula (I-B), (I-C) or (I-C1), examples of Rₐ include H, methyl, ethyl, prop-2-yn-1-yl (—CH₂—C≡CH), prop-2-en-1-yl (—CH₂—CH═CH₂), morpholino, benzyl, —CH₂CN, —CH₂CH₂N(CH₃)₂, or

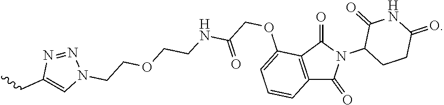

In preferred compounds of Formula (I-B) or (I-C), R₁ is propyl and R₇ is acetyloxy.

In a subset of compounds of Formula (I-B), Rₐ is prop-2-yn-1-yl, prop-2-yn-1-yl, or benzyl; R₁ is propyl; R₇ is acetyloxy (CH₃COO—); and [symbol] is a single bond.

In Formula (I-D), (I-E), (I-F), or (I-G), each of R_{d} and Rₑ can be methyl, but-3-yn-1-yl, t-butyl-OC(O)—NHCH₂—, (CH₃)₂NCH₂—, —NH₂, —CH₂NH₂, —CH₂CH₂NH₂, —CH₂CH₂N(CH₃)₂, —CH₂NHC(O)CH₃, —CH₂(CH₂)₄NH₂, —CH₂(CH₂)₄N(CH₃)₂,

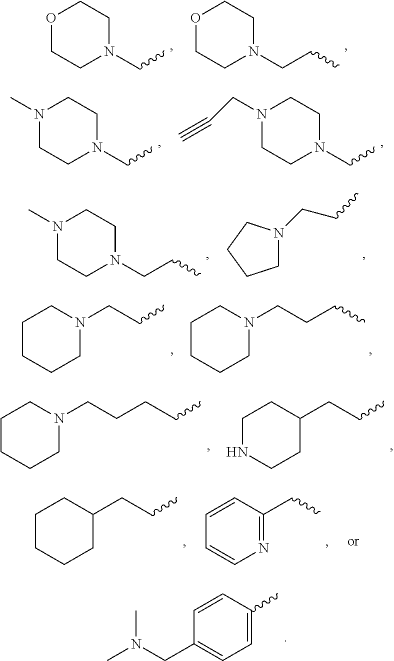

Each of R_{d} and Rₑ can also be

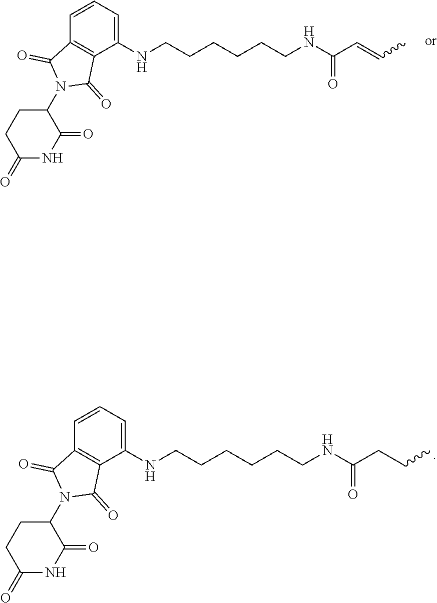

In a subset of the compounds of Formula (I-D), R₁ is propyl; R₇ is acetyloxy (CH₃COO—); R_{d} is

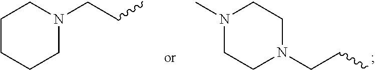

and [symbol] is a single bond.

In Formula (I-E) or (I-F), Rₐ is preferably H, methyl, ethyl, prop-2-yn-1-yl, prop-2-en-1-yl, morpholino, benzyl, —CH₂CN, —CH₂CH₂N(CH₃)₂, or

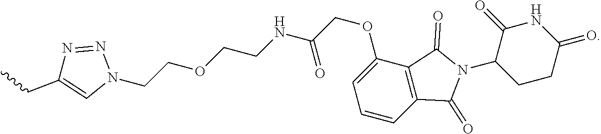

In a subset of the compounds of Formula (I-E), R₁ is propyl; R₇ is acetyloxy (CH₃COO—); Rₐ is prop-2-yn-1-yl; R_{d} is

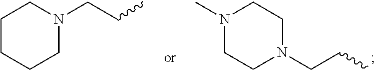

and [symbol] is a single bond.

Another aspect of this invention is a method of treating an inflammatory condition by administering to a subject in need thereof an effective amount of any compound described above. It further includes use of such a compound for treating an inflammatory condition or for the manufacture of a medicament for treating the inflammatory condition.

Also within the scope of this invention is a pharmaceutical composition comprising any of the compounds described above and a pharmaceutically acceptable carrier. As described above, the pharmaceutical composition is particularly useful in treating an inflammatory condition.

The table below shows structures of 72 exemplary compounds of the present invention, i.e., Compounds 1-72.

| | |
|---|---|
| 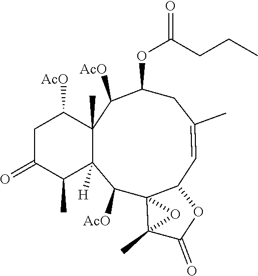 | 1 |
| 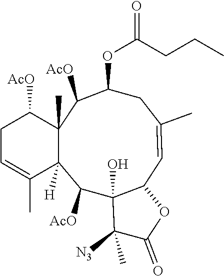 | 2 |
| 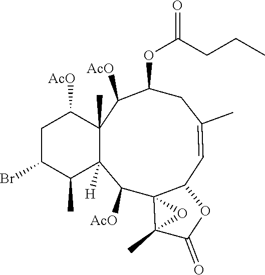 | 3 |
| 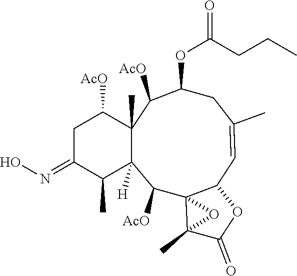 | 4 |
| 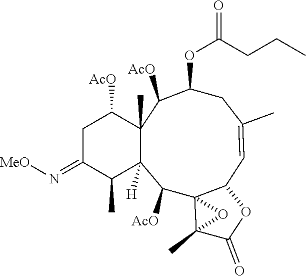 | 5 |
| 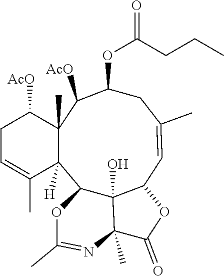 | 6 |
| 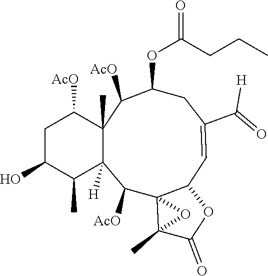 | 7 |
| 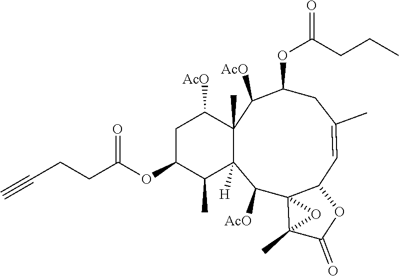 | 8 |
| 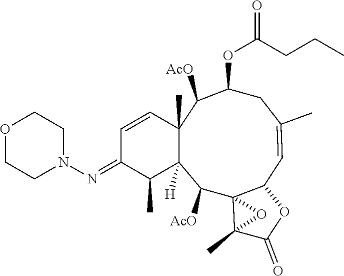 | 9 |
| 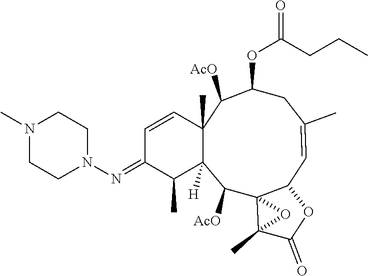 | 10 |
| 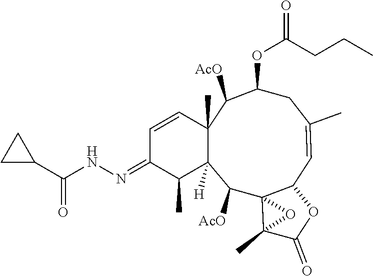 | 11 |
| 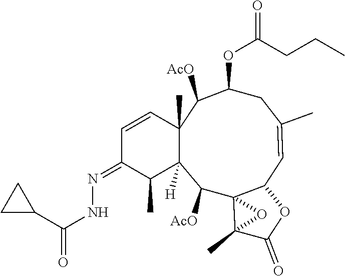 | 12 |
| 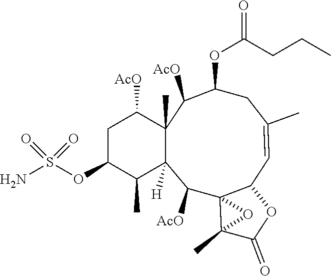 | 13 |
| 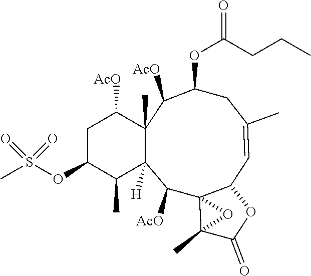 | 14 |
| 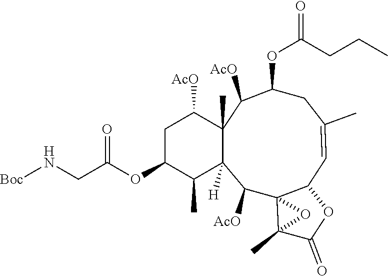 | 15 |
| 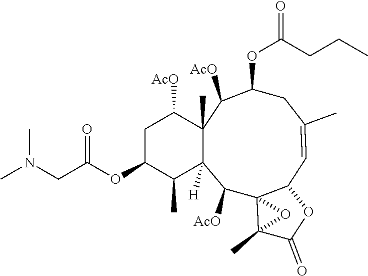 | 16 |
| 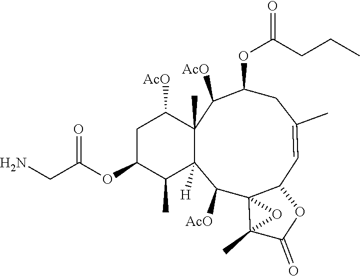 | 17 |
| 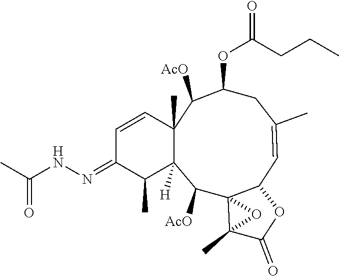 | 18 |
| 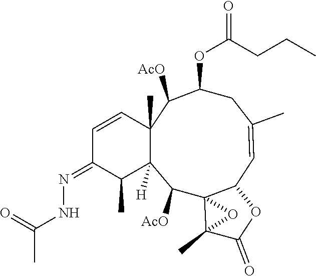 | 19 |
| 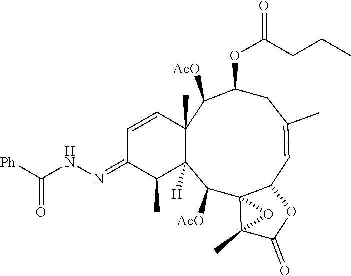 | 20 |
| 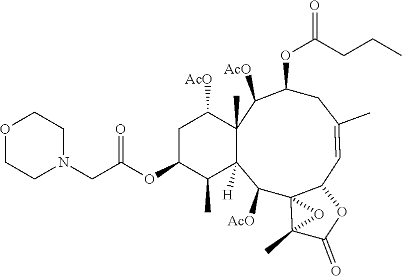 | 21 |
| 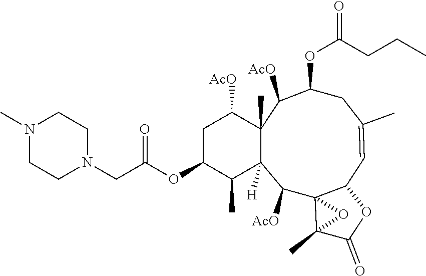 | 22 |
| 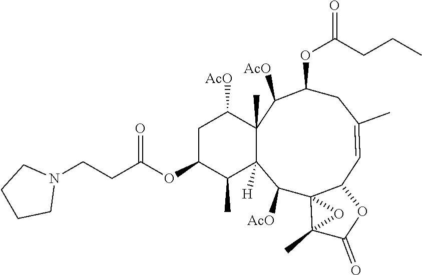 | 23 |
| 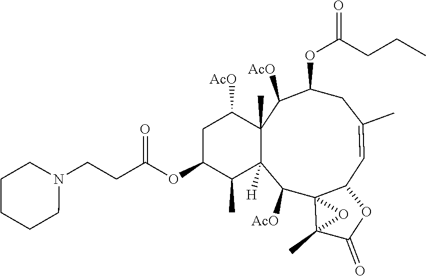 | 24 |
| 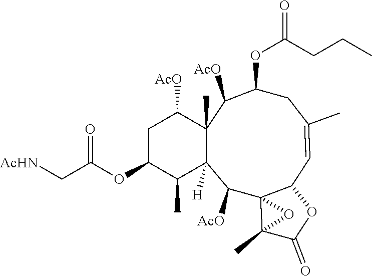 | 25 |
| 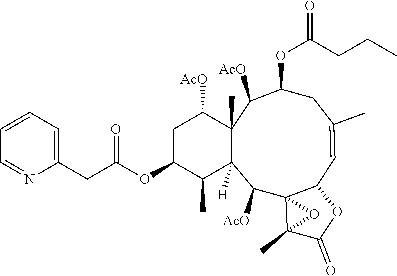 | 26 |
| 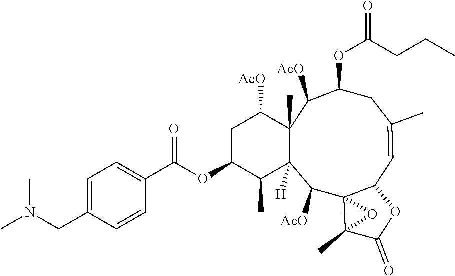 | 27 |
| 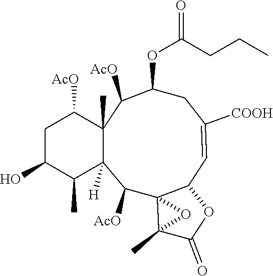 | 28 |
| 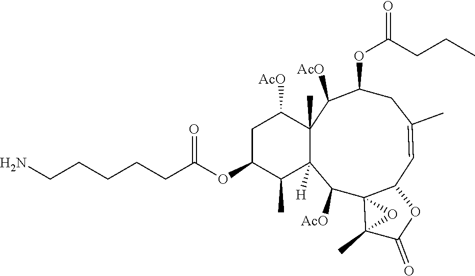 | 29 |
| 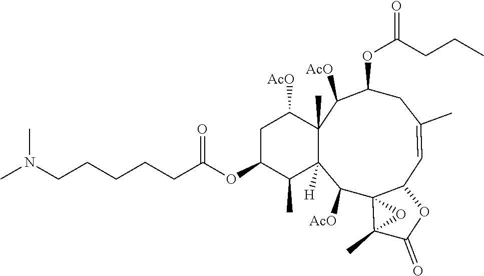 | 30 |
| 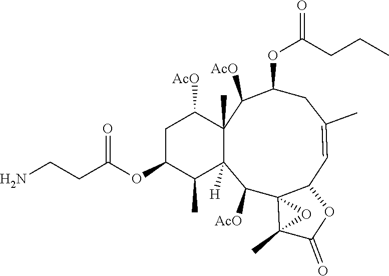 | 31 |
| 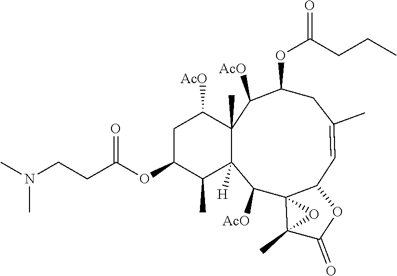 | 32 |
| 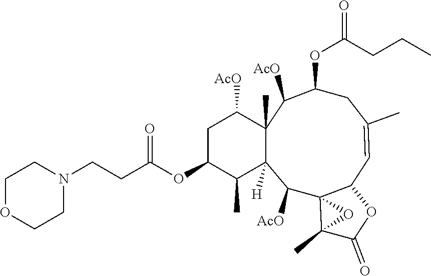 | 33 |
| 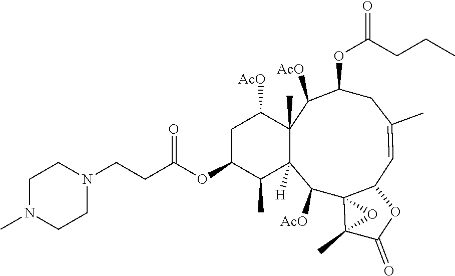 | 34 |
| 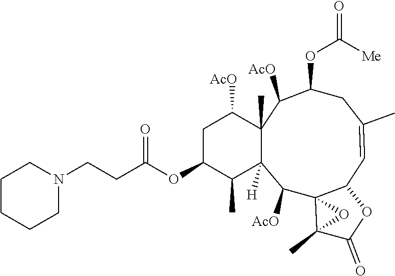 | 35 |
| 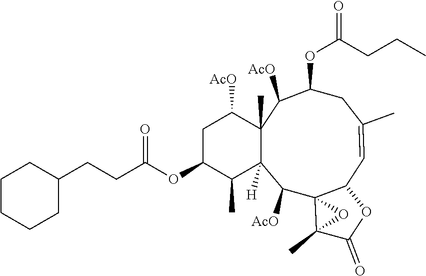 | 36 |
| 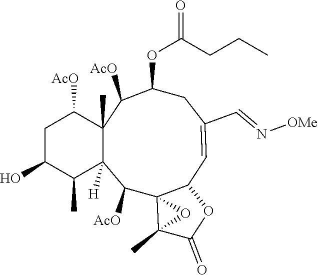 | 37 |
| 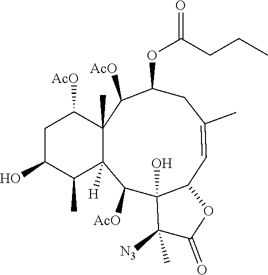 | 38 |
| 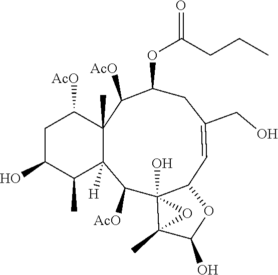 | 39 |
| 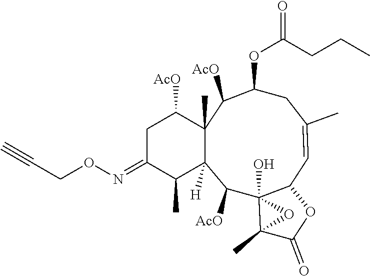 | 40 |
| 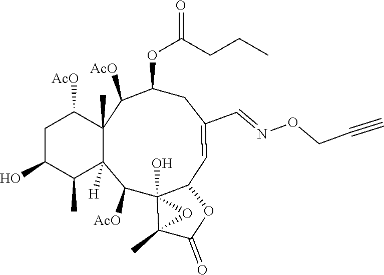 | 41 |
| 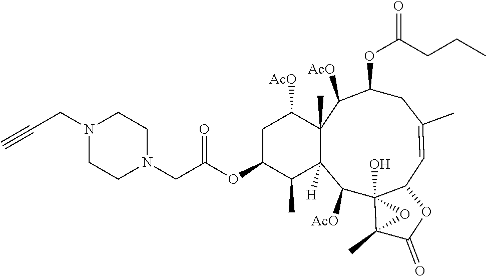 | 42 |
| 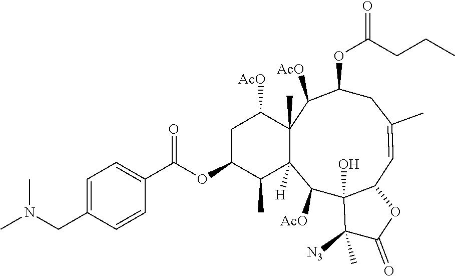 | 43 |
| 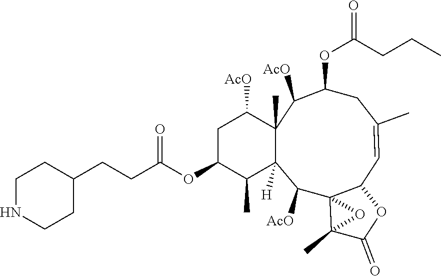 | 44 |
| 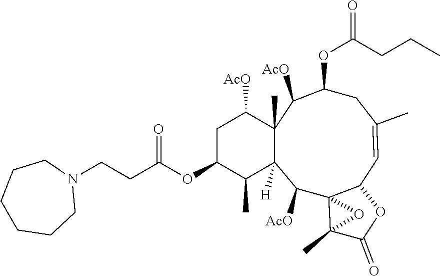 | 45 |
| 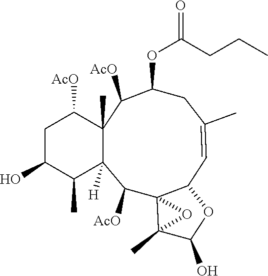 | 46 |
| 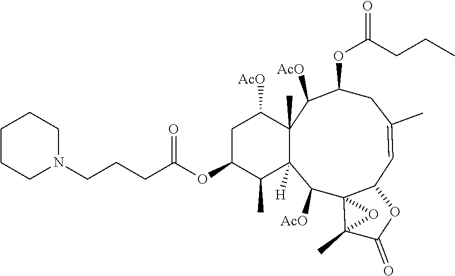 | 47 |
| 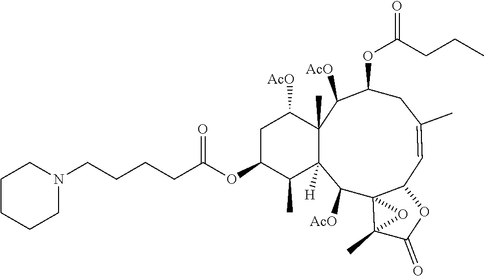 | 48 |
| 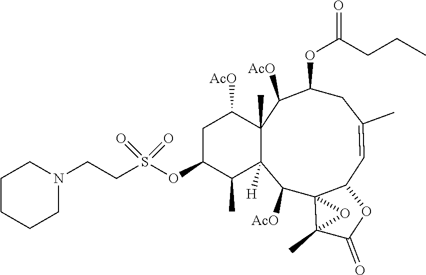 | 49 |
| 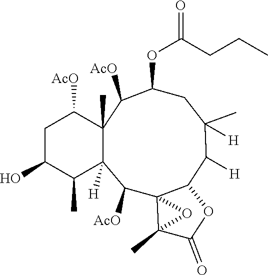 | 50 |
| 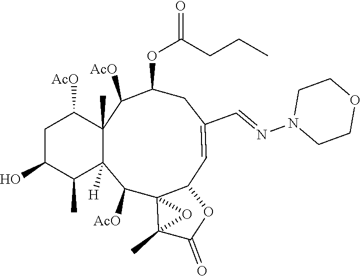 | 51 |
| 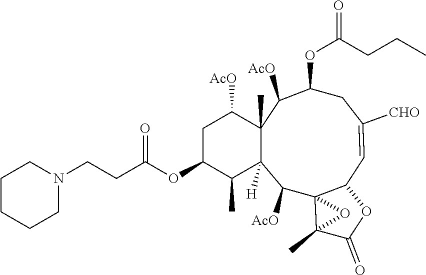 | 52 |
| 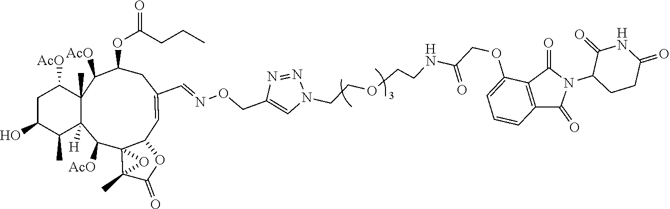 | 53 |
| 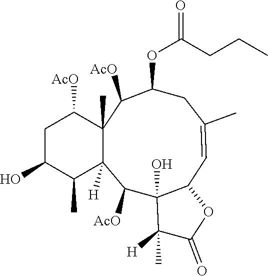 | 54 |
| 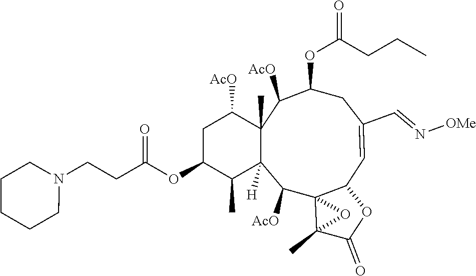 | 55 |
| 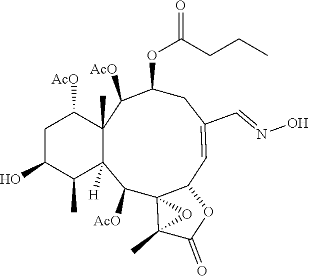 | 56 |
| 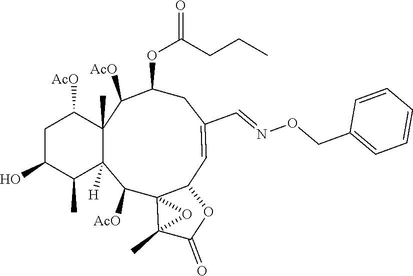 | 57 |
| 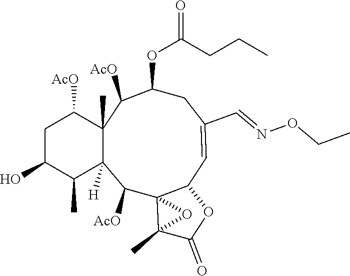 | 58 |
| 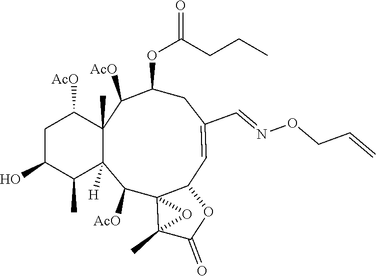 | 59 |
| 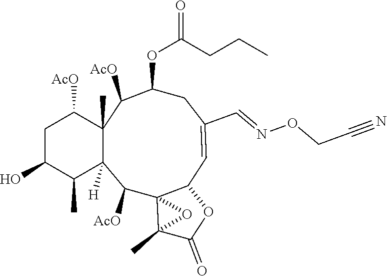 | 60 |
| 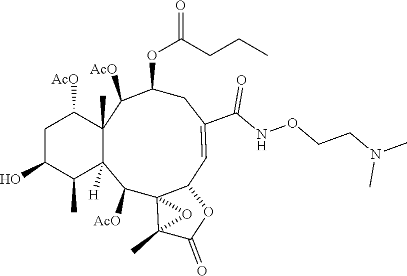 | 61 |
| 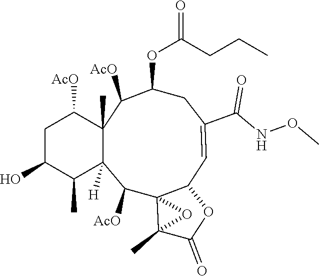 | 62 |
| 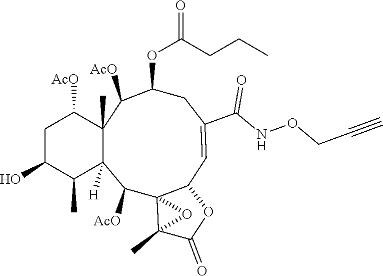 | 63 |
| 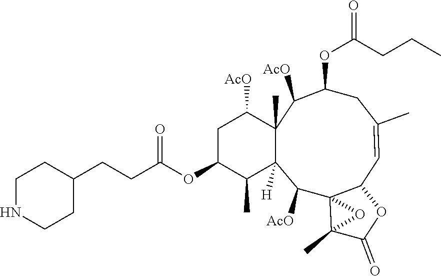 | 64 |
| 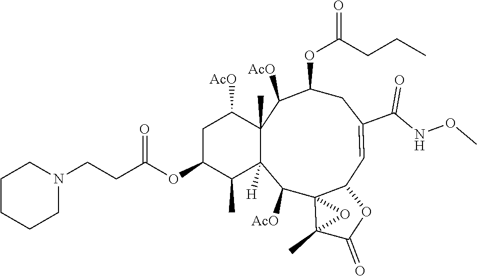 | 65 |
| 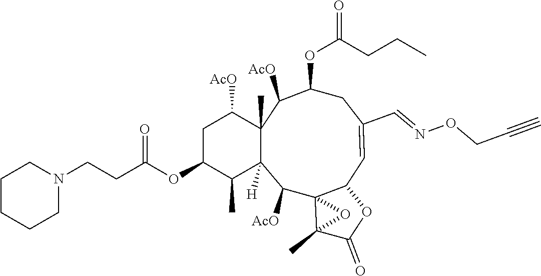 | 66 |
| 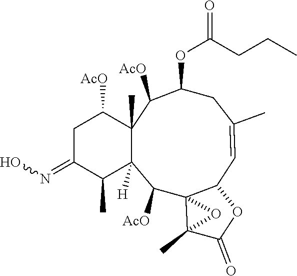 | 67 |
| 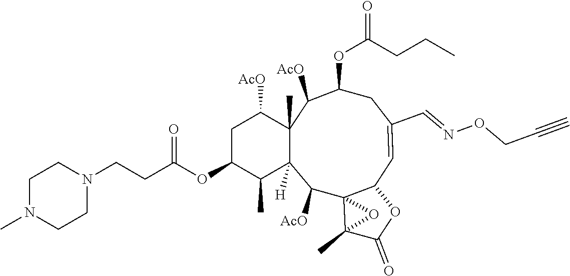 | 68 |
| 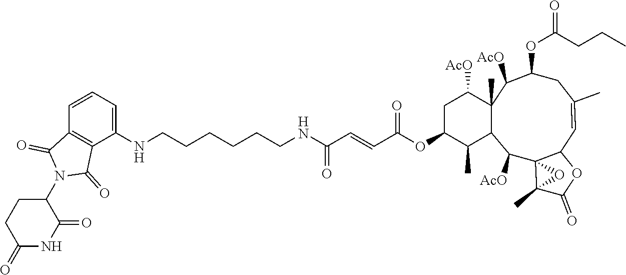 [SMILES: CCCC(=O)O[C@H]1C/C(C)=C\C2OC(=O)[C@@]3(C)O[C@]23[C@@H](OC(C)=O)C2[C@@H](C)[C@@H](OC(=O)/C=C/C(=O)NCCCCCCNc3cccc4c3C(=O)N(C3CCC(=O)NC3=O)C4=O)C[C@H](OC(C)=O)[C@]2(C)[C@H]1OC(C)=O] | 69 |
| 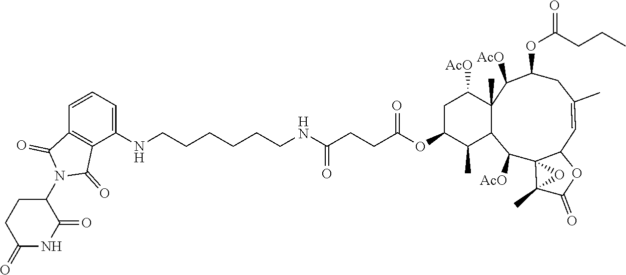 [SMILES: CCCC(=O)O[C@H]1C/C(C)=C\C2OC(=O)[C@@]3(C)O[C@]23[C@@H](OC(C)=O)C2[C@@H](C)[C@@H](OC(=O)CCC(=O)NCCCCCCNc3cccc4c3C(=O)N(C3CCC(=O)NC3=O)C4=O)C[C@H](OC(C)=O)[C@]2(C)[C@H]1OC(C)=O] | 70 |
| 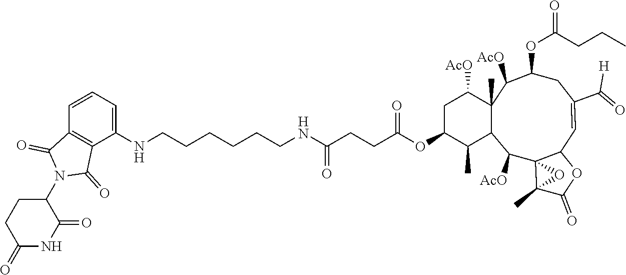 [SMILES: CCCC(=O)O[C@H]1C/C(C=O)=C\C2OC(=O)[C@@]3(C)O[C@]23[C@@H](OC(C)=O)C2[C@@H](C)[C@@H](OC(=O)CCC(=O)NCCCCCCNc3cccc4c3C(=O)N(C3CCC(=O)NC3=O)C4=O)C[C@H](OC(C)=O)[C@]2(C)[C@H]1OC(C)=O] | 71 |
| 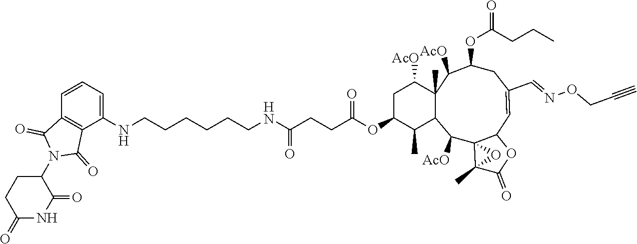 [SMILES: C#CCO/N=C/C1=C/C2OC(=O)[C@@]3(C)O[C@]23[C@@H](OC(C)=O)C2[C@@H](C)[C@@H](OC(=O)CCC(=O)NCCCCCCNc3cccc4c3C(=O)N(C3CCC(=O)NC3=O)C4=O)C[C@H](OC(C)=O)[C@]2(C)[C@@H](OC(C)=O)[C@@H](OC(=O)CCC)C1] | 72 |

Preferred compounds include Compounds 24, 34, 41, 57, 59, 66, 68, and 71.

The term “halo” herein refers to a fluoro, chloro, bromo, or iodo radical. Examples include a fluoro radical (F) and a bromo radical (Br). The term “amino” refers to a radical derived from amine, which is unsubstituted or mono-/di-substituted with alkyl, aryl, cycloalkyl, heterocycloalkyl, or heteroaryl. The term “aminoalkyl” refers to NH₂-alkyl, i.e., an alkyl that is substituted with at least one amino group. The term “alkylamino” refers to alkyl-NH—. Examples of aminoalkyl include aminomethyl and 2-aminoethyl. The term “acylamino” refers to —C(O)—NH—.

The term “alkyl” refers to a straight or branched hydrocarbon group, containing 1-20 carbon atoms (e.g., C₁₋₆) and a monovalent radical center derived by the removal of a hydrogen atom from a carbon atom of a parent alkane. Exemplary alkyl groups are methyl, ethyl, n-propyl, isopropyl, n-butyl, iso-butyl, tert-butyl, n-pentyl, and n-hexyl. The term “alkylcarbonyl” or “carbonyl” refers to alkyl-C(O)—. The term “haloalkyl” refers to alkyl substituted with one or more halo atoms. Examples include fluoromethyl, difluoromethyl, trifluoromethyl, fluoroethyl (e.g., 1-fluoroethyl and 2-fluoroethyl), difluoroethyl (e.g., 1,1-, 1,2-, and 2,2-difluoroethyl), and trifluoroethyl (e.g., 2,2,2-trifluoroethyl).

The term “alkoxy” refers to an —O-alkyl group. Examples are methoxy, ethoxy, propoxy, and isopropoxy. Alkoxy also includes haloalkoxy, namely, alkoxy substituted with one or more halogens, e.g., —O—CH₂Cl and —O—CHClCH₂Cl.

The term “formyl” refers to a —C(O)H group. The term “carboxyl” refers to a —C(O)—OH group. The term “carboxylate” refers to a —O—C(O)-alkyl group. The term ‘acetyloxy” refers to a —OC(O)CH₃ group. The term “oxo” refers to a ═O group.

The term “cycloalkyl” refers to a nonaromatic, saturated or unsaturated monocyclic, bicyclic, tricyclic, or tetracyclic hydrocarbon group containing 3 to 12 carbons (e.g., C₃₋₆ and C₃₋₁₀). Examples are cyclopropyl, cyclobutyl, cyclopentyl, cyclopentenyl, cyclohexyl, cyclohexenyl, cycloheptyl, and cyclooctyl. The term “heterocycloalkyl” refers to a nonaromatic, saturated or unsaturated, 3-8 membered monocyclic, 8-12 membered bicyclic, or 11-14 membered tricyclic ring system having one or more heteroatoms (e.g., O, N, P, and S). Examples include aziridinyl, azetidinyl, pyrrolidinyl, dihydrofuranyl, tetrahydrofuranyl, tetrahydropyranyl, tetrahydrothiophenyl, tetrahydro-2-H-thiopyran-1,1-dioxidyl, piperazinyl, piperidinyl, morpholinyl, imidazolidinyl, azepanyl, dihydrothiadiazolyl, dioxanyl, and quinuclidinyl. Both “cycloalkyl” and “heterocycloalkyl” also includes fused, bridged, and spiro ring systems.

The term “alkenyl” refers to a straight or branched, monovalent, unsaturated aliphatic chain having 2 to 20 carbon atoms (e.g., C₂₋₄, C₂₋₆, and C₂₋₁₀) and one or more carbon-carbon double bonds. Examples are ethenyl (also known as vinyl), 1-methylethenyl, 1-methyl-1-propenyl, 1-butenyl, 1-hexenyl, 2-methyl-2-propenyl, 1-propenyl, 2-propenyl, 2-butenyl, and 2-pentenyl. The term “alkenylene” refers to a straight or branched, bivalent, unsaturated aliphatic chain having 2 to 20 carbon atoms (e.g., C₂₋₄, C₂₋₆, and C₂₋₁₀) and one or more carbon-carbon double bonds. The term “alkenylalkyl” refers to an alkyl group substituted with a alkenyl group.

The term “alkynyl” refers to a straight or branched aliphatic chain having 2 to 20 carbon atoms (e.g., C₂₋₄, C₂₋₆, and C₂₋₁₀) and one or more carbon-carbon triple bonds. Examples are ethynyl, 2-propynyl, 2-butynyl, 3-methylbutnyl, and 1-pentynyl. The term “alkynylene” refers to a straight or branched, bivalent, unsaturated aliphatic chain having 2 to 20 carbon atoms (e.g., C₂₋₄, C₂₋₆, and C₂₋₁₀) and one or more carbon-carbon triple bonds. The term “alkynylalkyl” refers to an alkyl group substituted with an alkynyl group.

The term “aryl” refers a 6-carbon monocyclic, 10-carbon bicyclic, 14-carbon tricyclic aromatic ring system wherein each ring can have one or more (e.g., 1 to 10, 1 to 5, and 1 to 3) substituents. Examples include phenyl, biphenyl, 1- or 2-naphthyl, 1,2-dihydronaphthyl, 1,2,3,4-tetrahydronaphthyl, indenyl, and indanyl. The term “aralkyl” refers to alkyl substituted with an aryl group.

The term “heteroaryl” refers to an aromatic 5-8 membered monocyclic, 8-12 membered bicyclic, or 11-14 membered tricyclic ring system having one or more heteroatoms (e.g., O, N, P, and S). Examples include pyridinyl, pyrimidinyl, quinolinyl, isoquinolinyl, indolyl, benzimidazolyl, benzoxazolyl, benzothiophenyl, benzofuranyl, pyrazolyl, triazolyl, oxazolyl, thiadiazolyl, tetrazolyl, oxazolyl, isoxazolyl, carbazolyl, furyl, imidazolyl, thienyl, thiazolyl, and benzothiazolyl. The term “heterocyclylalkyl” refers to an alkyl group substituted with a heterocycloalkyl or heteroaryl group.

Alkyl, alkoxyl, alkenyl, alkenylalkyl, alkynyl, alkynylalkyl, carbonyl, alkylcarbonyl, carboxylate, amino, aminoalkyl, alkylamino, acylamino, cycloalkyl, heterocycloalkyl, aryl, aralkyl, heteroaryl, heterocyclylalkyl, and heteroaryl mentioned herein include both substituted and unsubstituted moieties, unless specified otherwise. Examples of a substituent include hydroxyl (OH), halo (e.g., F and Cl), amino (NH₂), cyano (CN), nitro (NO₂), alkyl, alkenyl, alkynyl, alkoxy, cycloalkyl, acylamino, alkylamino, aminoalkyl, haloalkyl (e.g., trifluoromethyl), heterocycloalkyl, alkoxycarbonyl, amido, carboxy (COOH), alkanesulfonyl, alkylcarbonyl, alkenylcarbonyl, carbamido, carbamyl, carboxyl, thioureido, thiocyanato, sulfonamido, aryl, arylamino, aralkyl, and heteroaryl. All substitutes can be further substituted.

The term “compound”, when referring to a compound of this invention, also includes its salts, solvates, and prodrugs. The pharmaceutically acceptable salts include those listed in Handbook of Pharmaceutical Salts: Properties, Selection and Use, 2^{nd} Revised Edition, P. H. Stahl and C. G. Wermuth (Eds.), Wiley-VCH, New York, (2011). In addition to pharmaceutically acceptable salts, other salts are contemplated in the invention. They may serve as intermediates in the purification of compounds or in the preparation of other pharmaceutically acceptable salts, or are useful for identification, characterization or purification of compounds of the invention. A solvate refers to a complex formed between an active compound and a pharmaceutically acceptable solvent. Examples of a pharmaceutically acceptable solvent include water, ethanol, isopropanol, ethyl acetate, acetic acid, and ethanolamine. A prodrug refers to a compound that, after administration, is metabolized into a pharmaceutically active drug. Examples of a prodrug include esters and other pharmaceutically acceptable derivatives.

The compounds of the present invention may contain one or more non-aromatic double bonds or asymmetric centers. Each of them occurs as a racemate or a racemic mixture, a single R enantiomer, a single S enantiomer, an individual diastereomer, a diastereometric mixture, a cis-isomer, or a trans-isomer. Compounds of such isomeric forms are within the scope of this invention. They can be present as a mixture or can be isolated using chiral synthesis or chiral separation technologies.

The present invention also features use of one or more of the above-described compounds for treating inflammatory conditions.

Inflammatory conditions or other disease conditions treatable by the current invention include autoimmune diseases, interferonopathies, cancers, neurological disorders, hepatitis B, age-related degeneration, viral infections, and cardiovascular diseases.

The term “treating” or “treatment” refers to administering one or more of the compounds to a subject with the purpose to confer a therapeutic effect, e.g., to slow, interrupt, arrest, control, or stop of the progression of an existing disorder and/or symptoms thereof, but does not necessarily indicate a total elimination of all symptoms. “An effective amount” refers to the amount of a compound that is required to confer the therapeutic effect. Effective doses will vary, as recognized by those skilled in the art, depending on the types of symptoms treated, route of administration, excipient usage, and the possibility of co-usage with other therapeutic treatment.

The term “subject” refers to an animal including human or non-human, such as a mammal. A human is a preferred subject.

A compound of this invention may be administered alone or in the form of a pharmaceutical composition with pharmaceutically acceptable carriers, diluents or excipients. Such pharmaceutical compositions and processes for making the same are known in the art (See, e.g., Remington: The Science and Practice of Pharmacy, A. Adejare, Editor, 23rd Edition., Academic Press, 2020).

To practice the method of the present invention, a composition or a kit containing one or more of the above-described compounds can be administered alone or co-administered with at least one other pharmacologically active substance simultaneously, concurrently, sequentially, successively, alternately, or separately. Simultaneous administration, also referring to as concomitant administration, includes administration at substantially the same time. Concurrent administration includes administering the active agents within the same general time period, for example on the same day(s) but not necessarily at the same time. Alternate administration includes administration of one agent during a time period, for example over the course of a few days or a week, followed by administration of the other agent(s) during a subsequent period of time, for example over the course of a few days or a week, and then repeating the pattern for one or more cycles. Sequential or successive administration includes administration of one agent during a first time period (for example over the course of a few days or a week) using one or more doses, followed by administration of the other agent(s) during a second and/or additional time period (for example over the course of a few days or a week) using one or more doses. An overlapping schedule may also be employed, which includes administration of the active agents on different days over the treatment period, not necessarily according to a regular sequence. Variations on these general guidelines may also be employed, e.g., according to the agents used and the condition of the subject.

The elements of the combinations of this invention may be administered (whether dependently or independently) by methods customary to the skilled person, e.g., by oral, enteral, parenteral, nasal, vaginal, rectal, or topical routes of administration and may be formulated, alone or together, in suitable dosage unit formulations containing conventional non-toxic pharmaceutically acceptable carriers, excipients and/or vehicles appropriate for each route of administration.

The term “parenteral” as used herein refers to subcutaneous, intracutaneous, intravenous, intraperitoneal, intramuscular, intraarticular, intraarterial, intrasynovial, intrasternal, intrathecal, intralesional, or intracranial injection, as well as any suitable infusion technique.

A composition for oral administration can be any orally acceptable dosage form including capsules, tablets, emulsions and aqueous suspensions, dispersions, and solutions. In the case of tablets, commonly used carriers include lactose and corn starch. Lubricating agents, such as magnesium stearate, are also typically added. For oral administration in a capsule form, useful diluents include lactose and dried corn starch. When aqueous suspensions or emulsions are administered orally, the active ingredient can be suspended or dissolved in an oily phase combined with emulsifying or suspending agents. If desired, certain sweetening, flavoring, or coloring agents can be added.

A nasal aerosol or inhalation composition can be prepared according to techniques well known in the art of pharmaceutical formulation. For example, such a composition can be prepared as a solution in saline, employing benzyl alcohol or other suitable preservatives, absorption promoters to enhance bioavailability, fluorocarbons, and/or other solubilizing or dispersing agents.

A composition having one or more of the above-described compounds can also be administered in the form of suppositories for rectal administration.

The carrier in the pharmaceutical composition must be “acceptable” in the sense that it is compatible with the active ingredient of the composition (and preferably, capable of stabilizing the active ingredient) and not deleterious to the subject to be treated. One or more solubilizing agents can be utilized as pharmaceutical excipients for delivery of an active compound. Examples include colloidal silicon oxide, magnesium stearate, cellulose, sodium lauryl sulfate, and D&C Yellow #10.

The details of one or more embodiments of the invention are set forth in the description below. Other features, objects, and advantages of the invention will be apparent from the description and from the claims.

### DETAILED DESCRIPTION

The present invention is based on a surprising discovery that the compounds of Formula (I) reproduced below are effective in inhibiting inflammation activity and treating disorders associated therewith.

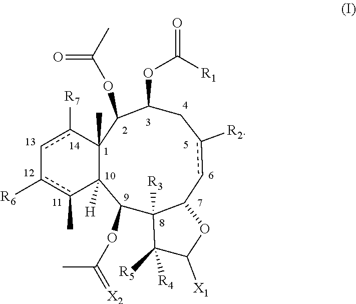

Variables R₁-R₇, X₁, and X₂ are defined above.

Formula (I) shows a bicyclo[8.4.0]tetradecane ring system as a core structure. The carbon atoms on the ring are numbered above for easy reference.

A subset of the compounds of Formula (I) is represented by Formula (I-A) as follows:

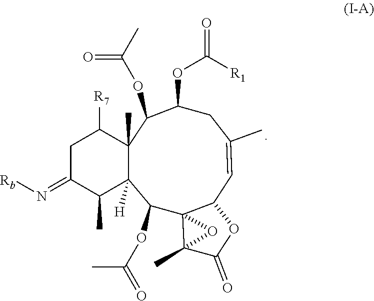

Another subset of the compounds of Formula (I) is represented by Formula (I-B) as shown below:

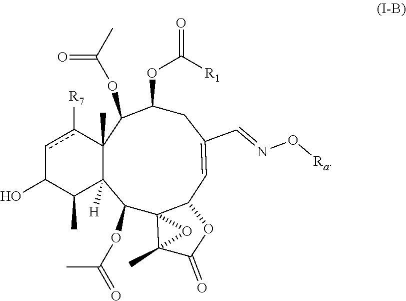

One more subset of the compounds of Formula (I) is represented by Formula (I-C):

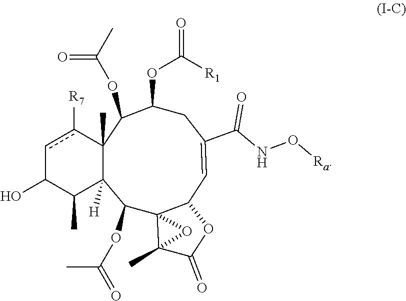

Still another subset of the compounds of Formula (I) is represented by Formula (I-D):

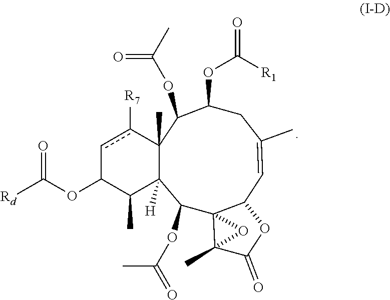

Yet another subset of the compounds of Formula (I) is represented by Formula (I-E):

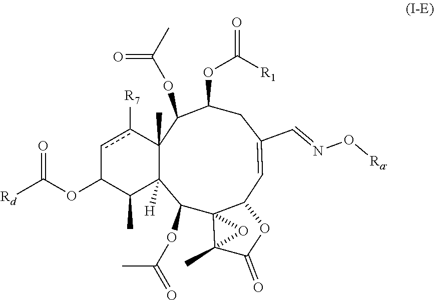

Further subset of the compounds of Formula (I) is represented by Formula (I-F):

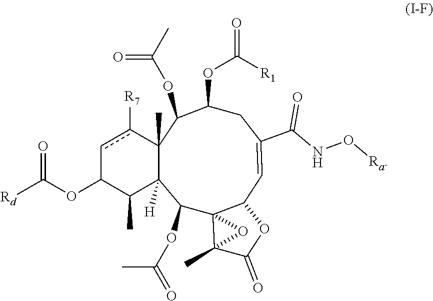

One more subset of the compounds of Formula (I) is represented by Formula (I-G):

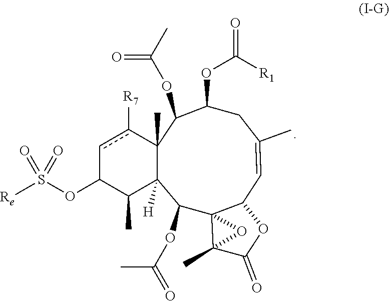

In any of Formulas (I-A) to (I-G) above, variables R₁, R₇, Rₐ, R_{b}, R_{d}, and Rₑ are defined above.

The compounds of Formula (I) can be prepared by synthetic methods well known in the art. See, e.g., R. Larock, Comprehensive Organic Transformations (3^{rd} Ed., John Wiley and Sons 2018); P. G. M. Wuts and T. W. Greene, Greene's Protective Groups in Organic Synthesis (4^{th} Ed., John Wiley and Sons 2007); L. Fieser and M. Fieser, Fieser and Fieser's Reagents for Organic Synthesis (John Wiley and Sons 1994); and L. Paquette, ed., Encyclopedia of Reagents for Organic Synthesis (2^{nd} ed., John Wiley and Sons 2009) and subsequent editions thereof.

As illustrated below, these compounds can be prepared by one of the following Procedures A, B, and C.

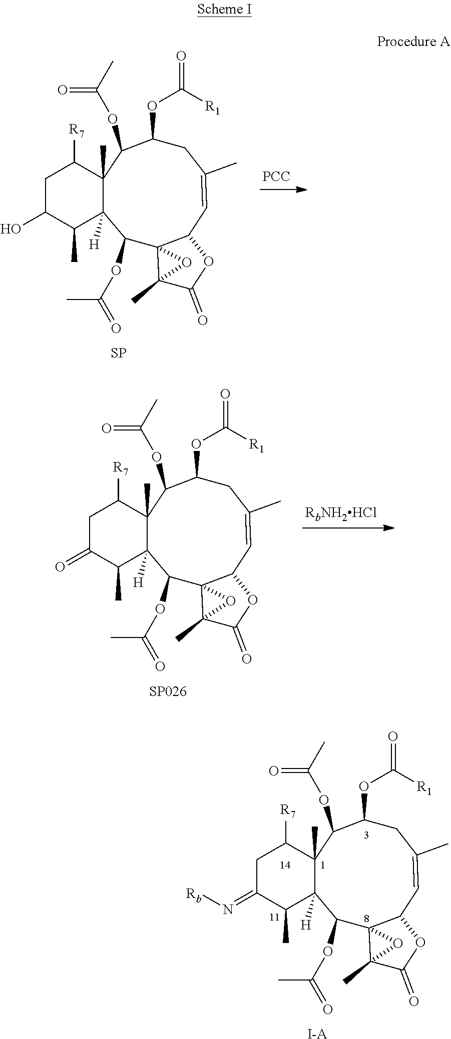

Provided below is an exemplary procedure. Under an ambient atmosphere (e.g., 20° C.), a 20-mL vial with a stir bar is charged with 1 molar equivalent Compound SP (e.g., ExcB, 182.7 mg, 0.307 mmol) and dried dichloromethane (2 mL), followed by the addition of pyridinium chlorochromate (“PCC”, 132.4 mg, 0.614 mmol, 2 equiv., commercially available from Millipore Sigma, Burlington, MA). The resultant mixture is stirred at room temperature (e.g., 20° C.) for 3 hours. It is then filtered through a short pad of diatomaceous earth (Celite®, MilliporeSigma) and rinsed with dichloromethane. The filtrate is collected and concentrated by rotary evaporation. The residue can be purified by flash column chromatography on silica gel (EtOAc:n-Hexane from 0:1 to 1:1) to afford Compound SP026 at a quantitative yield, usually as a white solid.

Compound 1 (1 equiv.) is then mixed with O-methylhydroxylamine hydrochloride (3.8 mg, 0.0449 mmol, 1.5 equiv.) in dichloromethane/pyridine (0.3 mL, ratio 14:1), and subsequently stirred at room temperature for 0.5 hours. Purification with flash column chromatography (EtOAc:n-Hexane from 0:1 to 1:1) affords a compound of Formula (I-A) at an excellent yield (e.g., 90% or greater), often as a colorless oil.

The following compounds were prepared using Procedure A: Compounds 1, 4, 5, 40, and 67.

Compound 1: ¹H NMR (600 MHz, CDCl₃, 22° C.) δ 5.78 (s, 1H), 5.35 (s, 2H), 5.31-5.23 (m, 2H), 5.17 (dd, J=9.8, 4.0 Hz, 1H), 3.61 (dd, J=15.7, 4.9 Hz, 1H), 3.25 (dd, J=9.3, 5.7 Hz, 1H), 3.01-2.93 (m, 1H), 2.86 (t, J=13.3 Hz, 1H), 2.78 (dd, J=16.5, 4.0 Hz, 1H), 2.31 (s, 3H), 2.24 (t, J=7.4 Hz, 2H), 2.18 (s, 3H), 2.11 (s, 3H), 2.10-2.06 (m, 1H), 1.81 (s, 3H), 1.66-1.60 (m, 2H), 1.59 (s, 3H), 1.23 (d, J=7.5 Hz, 3H), 1.14 (s, 3H), 0.94 (t, J=7.4 Hz, 3H). HRMS-ESI (m/z) calculated for C₃₀H₄₀O₁₂Na [M+Na]⁺ 615.2412, found 615.2416.

Compound 4: HRMS-FAB (m/z) calculated for C₃₀H₄₁NO₁₂Na [M+Na]⁺ 630.2521, found 630.2514.

Compound 5: HRMS-ESI (m/z) calculated for C₃₁H₄₃NO₁₂Na [M+Na]⁺ 644.2678, found 644.2669.

Compound 40: MS-ESI (m/z) calculated for C₃₃H₄₄NO₁₂ [M+H]⁺ 646.2864, found 646.3.

Compound 67: HRMS-FAB (m/z) calculated for C₃₀H₄₁NO₁₂Na [M+Na]+ 630.2521, found 630.2514.

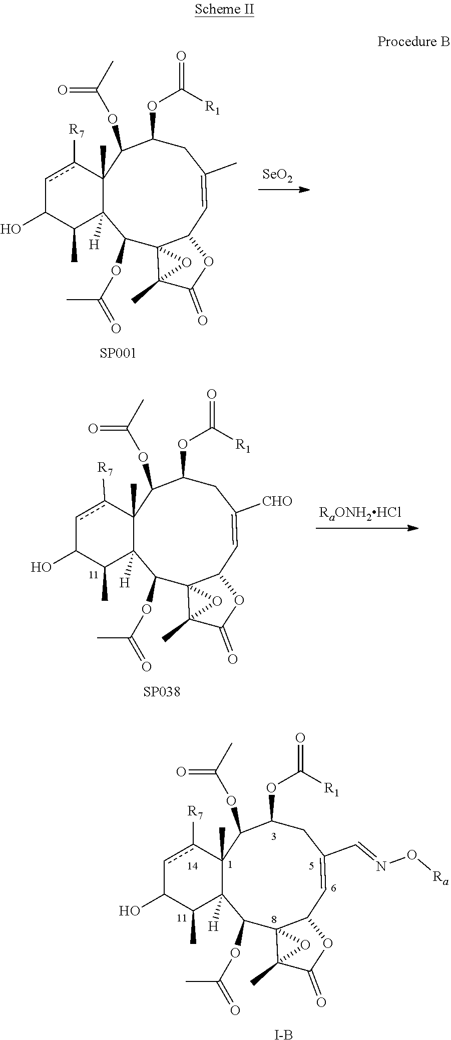

At room temperature, a 4-mL vial equipped with a stir bar is charged with 1 molar equivalent Compound SP001 (e.g., ExcB, 182.7 mg, 0.307 mmol), SeO₂ (100.9 mg, 0.91 mmol, 10 equiv., MilliporeSigma), and 1,4-dioxane (1 mL, MilliporeSigma). The resultant mixture is stirred at 80° C. for 22 hours, cooled to room temperature, and then filtered through a short pad of Celite®. The filtrate is concentrated under reduced pressure to give a residue, which is purified by flash column chromatography (EtOAc:n-Hexane from 0:1 to 2:3 then to 1:1) to afford Compound SP038 at a high yield (e.g., 80% or greater), usually as a white solid.

Compound SP038 (1 equiv.) is then mixed with 1.5 equivalents O-methylhydroxylamine hydrochloride in dichloromethane/pyridine (0.3 mL, ratio 14:1), and subsequently stirred at room temperature for 0.5 hours. Purification with flash column chromatography (EtOAc:n-Hexane from 0:1 to 1:1) affords a compound of Formula I-B at a high yield (e.g., 80% or greater) often as a colorless oil.

The following compounds were prepared using Procedure B: Compounds 7, 37, 41, 51, and 56-60.

Compound 7: ¹H NMR (600 MHz, acetone-d₆, −40° C.) δ 9.69 (d, J=1.6 Hz, 1H), 6.88 (dd, J=7.5, 2.1 Hz, 1H), 5.96 (dd, J=7.4, 1.5 Hz, 1H), 5.71 (dd, J=7.6, 2.7 Hz, 1H), 5.64 (d, J=10.4 Hz, 1H), 5.19 (d, J=2.5 Hz, 1H), 4.59-4.51 (m, 1H), 4.20-4.11 (m, 1H), 3.96-3.79 (m, 2H), 2.87 (dd, J=10.4, 5.0 Hz, 1H), 2.57-2.50 (m, 1H), 2.46 (d, J=15.1 Hz, 1H), 2.42 (s, 3H), 2.21 (s, 3H), 2.22-2.05 (m, 2H), 1.99 (s, 3H), 1.89-1.81 (m, 1H), 1.79-1.71 (m, 1H), 1.55 (s, 3H), 1.54-1.49 (m, 2H), 1.03 (d, J=7.2 Hz, 3H), 0.90 (t, J=7.4 Hz, 3H), 0.79 (s, 3H). HRMS-ESI (m/z) calculated for C₃₀H₄₀O₁₃Na [M+Na]⁺ 631.2361, found 631.2370.

Compound 37: ¹H NMR (600 MHz, acetone-d₆, −40° C.) δ 7.97 (s, 1H), 6.06-5.99 (m, 1H), 5.92 (d, J=6.0 Hz, 1H), 5.83 (d, J=7.4 Hz, 1H), 5.61 (d, J=10.3 Hz, 1H), 5.17 (s, 1H), 4.58 (s, 1H), 4.22-4.15 (m, 1H), 3.97 (dd, J=15.7, 7.3 Hz, 1H), 3.91-3.83 (m, 1H), 3.75 (s, 3H), 2.98 (dd, J=10.4, 5.0 Hz, 1H), 2.57-2.48 (m, 2H), 2.41 (s, 3H), 2.21 (s, 3H), 2.19-2.08 (m, 2H), 2.05 (s, 3H), 1.91-1.80 (m, 1H), 1.80-1.73 (m, 1H), 1.58-1.46 (m, 2H), 1.53 (s, 3H), 1.04 (d, J=7.2 Hz, 3H), 0.89 (t, J=7.4 Hz, 3H), 0.81 (s, 3H). HRMS-ESI (m/z) calculated for C₃₁H₄₃NO₁₃Na [M+Na]⁺ 660.2627, found 660.2634.

Compound 41: ¹H NMR (600 MHz, acetone-d₆, −40° C.) δ 8.04 (s, 1H), 6.09 (dd, J=7.5, 2.0 Hz, 1H), 5.92 (dd, J=6.6, 2.0 Hz, 1H), 5.84 (d, J=7.4 Hz, 1H), 5.62 (d, J=10.4 Hz, 1H), 5.16 (d, J=2.5 Hz, 1H), 4.65 (dd, J=15.8, 2.5 Hz, 1H), 4.61-4.55 (m, 2H), 4.17 (d, J=4.0 Hz, 1H), 4.01 (dd, J=15.6, 6.7 Hz, 1H), 3.91-3.84 (m, 1H), 3.26 (t, J=2.4 Hz, 1H), 2.96 (dd, J=10.4, 5.1 Hz, 1H), 2.58-2.49 (m, 2H), 2.41 (s, 3H), 2.21 (s, 3H), 2.17-2.05 (m, 2H), 2.05 (s, 3H), 1.90-1.80 (m, 1H), 1.79-1.72 (m, 1H), 1.58-1.48 (m, 2H), 1.53 (s, 3H), 1.04 (d, J=7.3 Hz, 3H), 0.89 (t, J=6.0 Hz, 3H), 0.81 (s, 3H). HRMS-ESI (m/z) calculated for C₃₃H₄₃NO₁₃Na [M+Na]⁺ 684.2627, found 684.2620.

Compound 51: MS-ESI (m/z) calcd for C₃₄H₄₉N₂O₁₃ [M+H]⁺ 693.3235, found 693.3.

Compound 56: ¹H NMR (600 MHz, acetone-d₆, −40° C.) δ 10.81 (s, 1H), 7.97 (s, 1H), 5.98 (d, J=7.6 Hz, 1H), 5.91 (d, J=4.9 Hz, 1H), 5.83 (d, J=7.7 Hz, 1H), 5.60 (d, J=10.3 Hz, 1H), 5.21 (br s, 1H), 4.56 (br s, 1H), 4.19 (d, J=3.9 Hz, 1H), 3.92 (dd, J=15.4, 7.2 Hz, 1H), 3.89-3.84 (m, 1H), 3.03 (dd, J=10.4, 5.0 Hz, 1H), 2.57-2.49 (m, 2H), 2.41 (s, 3H), 2.20 (s, 3H), 2.14-2.05 (m, 1H), 2.02 (s, 3H), 2.03-1.93 (m, 1H), 1.89-1.80 (m, 1H), 1.79-1.73 (m, 1H), 1.52 (s, 3H), 1.51-1.40 (m, 2H), 1.03 (d, J=7.1 Hz, 3H), 0.84 (t, J=7.4 Hz, 3H), 0.80 (s, 3H). HRMS-ESI (m/z) calculated for C₃₀H₄₁NO₁₃Na [M+Na]⁺ 646.2470, found 646.2461.

Compound 57: MS-ESI (m/z) calculated for C₃₀H₄₁NO₁₃Na [M+Na]⁺ 646.2470, found 646.2461.

Compound 58: MS-ESI (m/z) calculated for C₃₂H₄₆NO₁₃ [M+H]⁺ 652.2969, found 652.3.

Compound 59: MS-ESI (m/z) calculated for C₃₃H₄₆NO₁₃ [M+H]⁺ 664.2969, found 664.3.

Compound 60: MS-ESI (m/z) calculated for C₃₂H₄₂N₂O₁₃Na [M+Na]⁺ 685.2585, found 685.2.

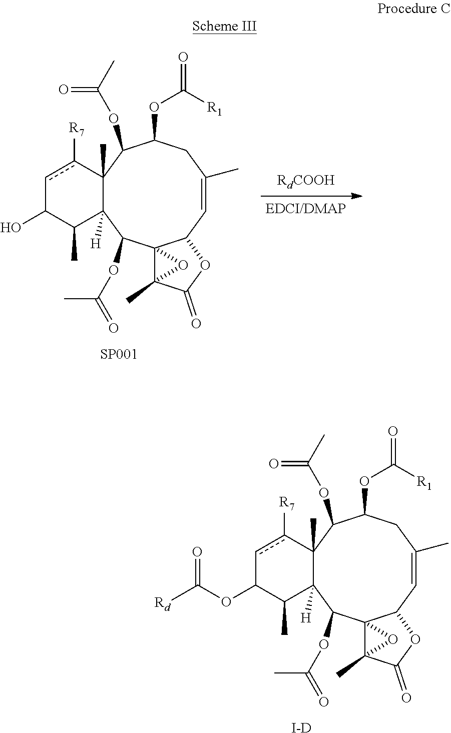

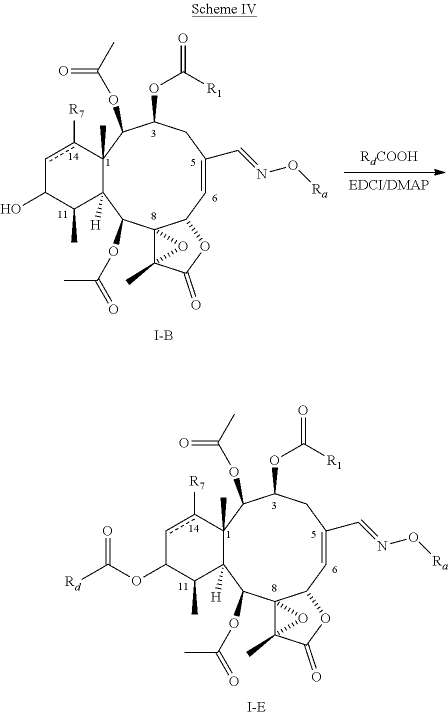

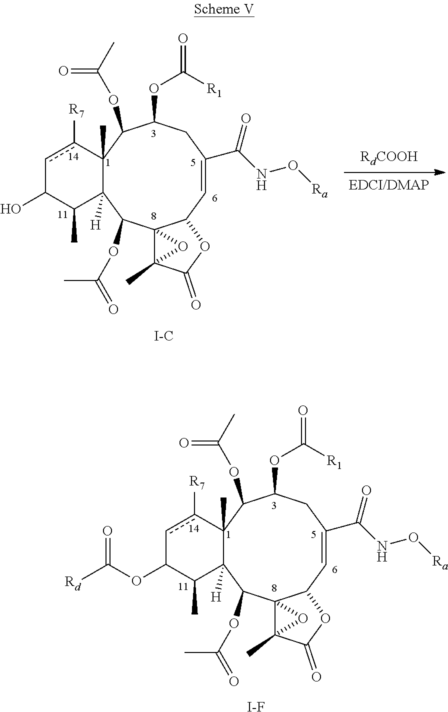

Schemes III, IV, and V above show preparation of a compound of one of Formula (I-D), (I-E), and (I-F). At room temperature, 1 molar equivalent Compound SP001 or a compound of Formula (I-B) or (I-C) is mixed with carboxylic acid R_{d}COOH (2 equiv., MilliporeSigma), dimethylamino-pyridine (“DMAP”, 0.3 equiv., MilliporeSigma), 1-ethyl-3-(3-dimethylaminopropyl)carbodi-imide (“EDCI”, 3 equiv., MilliporeSigma) and dried CH₂Cl₂ (0.5 mL) or dried dimethyl-formamide (0.5 mL, MilliporeSigma). The resultant mixture is stirred at room temperature for 18 hours, diluted with CH₂Cl₂, and then washed with water. The organic layer is collected and dried over anhydrous MgSO₄, filtered, and concentrated to obtain a residue, which is purified by flash column chromatography to afford a compound of corresponding Formula (I-D), (I-E), or (I-F).

The following compounds were prepared using Procedure C: Compounds 8, 15, 16, 17, 21-27, 29-36, 42, 44, 45, 47, 48, 52, 55, 64, 65, 66, and 68.

Compound 8: MS-ESI (m/z) calculated for C₃₅H₄₆O₁₀Na [M+Na]⁺ 697.2836, found 697.2.

Compound 15: ¹H NMR (600 MHz, acetone-d₆, −40° C.) δ 6.75 (X part of ABX system, J=6.3 Hz, 1H), 5.85 (dd, J=6.9, 1.8 Hz, 1H), 5.67 (d, J=7.3 Hz, 1H), 5.53 (d, J=10.4 Hz, 1H), 5.36 (dt, J=7.4, 1.9 Hz, 1H), 5.16 (d, J=2.4 Hz, 1H), 5.01 (ddd, J=12.5, 5.0, 3.6 Hz, 1H), 4.74-4.66 (m, 1H), 4.01 (dd, J=15.8, 7.3 Hz, 1H), 3.81, 3.78 (AB part of ABX system, J_{AB}=18.0 Hz, J_{AX}=6.6 Hz, J_{BX}=6.6 Hz, 2H), 3.17 (dd, J=10.4, 5.1 Hz, 1H), 2.68-2.60 (m, 1H), 2.41 (s, 3H), 2.23 (s, 6H), 2.22-2.06 (m, 4H), 1.95 (s, 3H), 1.90-1.85 (m, 1H), 1.55-1.46 (m, 2H), 1.50 (s, 3H), 1.37 (s, 9H), 1.10 (d, J=7.1 Hz, 3H), 0.86 (s, 3H), 0.85 (t, J=7.4 Hz, 3H). HRMS-ESI (m/z) calculated for C₃₇H₅₃NO₁₅Na [M+Na]⁺ 774.3307, found 774.3311.

Compound 16: ¹H NMR (600 MHz, acetone-d₆, −40° C.) δ 5.85 (dd, J=6.9, 1.7 Hz, 1H), 5.67 (d, J=7.3 Hz, 1H), 5.52 (d, J=10.4 Hz, 1H), 5.40-5.33 (m, 1H), 5.16 (d, J=2.4 Hz, 1H), 5.03 (ddd, J=12.4, 5.0, 3.6 Hz, 1H), 4.74-4.67 (m, 1H), 4.01 (dd, J=15.8, 7.3 Hz, 1H), 3.27, 3.24 (ABq, J=16.9 Hz, 2H), 3.16 (dd, J=10.4, 5.1 Hz, 1H), 2.67-2.60 (m, 1H), 2.41 (s, 3H), 2.30 (s, 6H), 2.23 (s, 3H), 2.23 (s, 3H), 2.22-2.07 (m, 4H), 1.95 (s, 3H), 1.91-1.85 (m, 1H), 1.55-1.46 (m, 2H), 1.50 (s, 3H), 1.09 (d, J=7.1 Hz, 3H), 0.86 (s, 3H), 0.85 (t, J=7.4 Hz, 3H). HRMS-ESI (m/z) calculated for C₃₄H₅₀NO₁₃ [M+H]⁺ 680.3277, found 680.3279.

Compound 17: HRMS-ESI (m/z) calculated for C₃₂H₄₆NO₁₃ [M+H]⁺ 652.2964, found 652.2966.

Compound 21: ¹H NMR (600 MHz, acetone-d₆, −40° C.) δ 5.88-5.82 (m, 1H), 5.67 (d, J=7.3 Hz, 1H), 5.52 (d, J=10.4 Hz, 1H), 5.37 (dt, J=7.4, 1.8 Hz, 1H), 5.16 (d, J=2.4 Hz, 1H), 5.02 (ddd, J=12.5, 5.0, 3.6 Hz, 1H), 4.70 (dd, J=4.1, 2.1 Hz, 1H), 4.01 (dd, J=15.8, 7.3 Hz, 1H), 3.79-3.40 (m, 4H), 3.24, 3.21 (ABq, J=17.0 Hz, 2H), 3.16 (dd, J=10.4, 5.2 Hz, 1H), 2.79-2.59 (m, 3H), 2.41 (s, 3H), 2.38-2.26 (m, 2H), 2.23 (s, 3H), 2.23 (s, 3H), 2.21-2.06 (m, 4H), 1.95 (s, 3H), 1.90-1.85 (m, 1H), 1.56-1.46 (m, 2H), 1.51 (s, 3H), 1.09 (d, J=7.2 Hz, 3H), 0.86 (s, 3H), 0.85 (t, J=7.4 Hz, 3H). HRMS-ESI (m/z) calculated for C₃₆H₅₂NO₁₄ [M+H]⁺ 722.3382, found 722.3388.

Compound 22: ¹H NMR (600 MHz, acetone-d₆, −40° C.) δ 5.85 (d, J=7.6 Hz, 1H), 5.67 (d, J=7.3 Hz, 1H), 5.52 (d, J=10.4 Hz, 1H), 5.36 (dd, J=7.3, 2.0 Hz, 1H), 5.16 (d, J=2.3 Hz, 1H), 5.07-4.98 (m, 1H), 4.70 (br s, 1H), 4.01 (dd, J=15.8, 7.3 Hz, 1H), 3.21, 3.18 (ABq, J=16.9 Hz, 2H), 3.17 (dd, J=10.6, 5.2 Hz, 1H), 2.77-2.66 (m, 2H), 2.68-2.58 (m, 3H), 2.41 (s, 3H), 2.30-2.15 (m, 3H), 2.23 (s, 6H), 2.14-2.01 (m, 3H), 2.12 (s, 3H), 1.99-1.91 (m, 2H) 1.95 (s, 3H), 1.91-1.84 (m, 1H), 1.53-1.47 (m, 2H), 1.50 (s, 3H), 1.09 (d, J=7.1 Hz, 3H), 0.86 (s, 3H), 0.85 (t, J=7.3 Hz, 3H). HRMS-ESI (m/z) calculated for C₃₇H₅₅N₂O₁₃ [M+H]⁺ 735.3699, found 735.3694.

Compound 23: ¹H NMR (600 MHz, acetone-d₆, −40° C.) δ 5.88-5.82 (m, 1H), 5.67 (d, J=7.3 Hz, 1H), 5.53 (d, J=10.3 Hz, 1H), 5.36 (d, J=7.3 Hz, 1H), 5.16 (d, J=2.4 Hz, 1H), 4.99 (ddd, J=12.4, 5.0, 3.6 Hz, 1H), 4.72-4.68 (m, 1H), 4.01 (dd, J=15.8, 7.2 Hz, 1H), 3.15 (dd, J=10.3, 5.1 Hz, 1H), 2.71-2.58 (m, 3H), 2.55-2.41 (m, 6H), 2.41 (s, 3H), 2.23 (s, 6H), 2.21-2.07 (m, 3H), 2.04-2.00 (m, 1H), 1.95 (s, 3H), 1.89-1.84 (m, 1H), 1.71-1.63 (m, 4H), 1.57-1.46 (m, 2H), 1.50 (s, 3H), 1.10 (d, J=7.1 Hz, 3H), 0.86 (s, 3H), 0.85 (t, J=7.3 Hz, 3H). HRMS-ESI (m/z) calculated for C₃₇H₅₄NO₁₃ [M+H]⁺ 720.3590, found 720.3598.

Compound 24: ¹H NMR (600 MHz, acetone-d₆, −40° C.) δ 5.88-5.82 (m, 1H), 5.67 (d, J=7.3 Hz, 1H), 5.53 (d, J=10.3 Hz, 1H), 5.16 (d, J=2.3 Hz, 1H), 5.38-5.34 (m, 1H), 5.00 (dt, J=12.4, 4.3 Hz, 1H), 4.70 (dd, J=3.9, 2.1 Hz, 1H), 4.73-4.67 (m, 1H), 4.01 (dd, J=15.8, 7.2 Hz, 1H), 3.14 (dd, J=10.3, 5.1 Hz, 1H), 2.94-2.80 (m, 2H), 2.69-2.61 (m, 1H), 2.57-2.42 (m, 4H), 2.41 (s, 3H), 2.23 (s, 3H), 2.23 (s, 3H), 2.22-2.00 (m, 5H), 1.95 (s, 3H), 1.90-1.83 (m, 1H), 1.81-1.74 (m, 2H), 1.66-1.35 (m, 7H), 1.50 (s, 3H), 1.04-1.17 (m, 1H), 1.10 (d, J=7.1 Hz, 3H), 0.86 (s, 3H), 0.85 (t, J=7.4 Hz, 3H). HRMS-ESI (m/z) calculated for C₃₈H₅₆NO₁₃ [M+H]⁺ 734.3746, found 734.3741.

Compound 25: MS-ESI (m/z) calcd for C₃₄H₄₈NO₁₄[M+H]⁺ 694.3075, found 694.3.

Compound 26: ¹H NMR (600 MHz, acetone-d₆, −40° C.) δ 8.52-8.46 (m, 1H), 7.77 (td, J=7.7, 1.8 Hz, 1H), 7.39 (d, J=7.8 Hz, 1H), 7.28 (ddd, J=7.6, 4.9, 1.1 Hz, 1H), 5.84 (dd, J=6.9, 1.8 Hz, 1H), 5.66 (d, J=7.3 Hz, 1H), 5.52 (d, J=10.4 Hz, 1H), 5.39-5.33 (m, 1H), 5.16 (d, J=2.4 Hz, 1H), 5.02 (ddd, J=12.4, 5.0, 3.6 Hz, 1H), 4.72-4.67 (m, 1H), 4.00 (dd, J=15.8, 7.3 Hz, 1H), 3.87, 3.84 (ABq, J=16.0 Hz, 2H), 3.16 (dd, J=10.4, 5.2 Hz, 1H), 2.68-2.60 (m, 1H), 2.40 (s, 3H), 2.22 (s, 3H), 2.19 (s, 3H), 2.19-2.06 (m, 2H), 2.03-1.99 (m, 1H), 1.94 (s, 3H), 1.91-1.86 (m, 1H), 1.55-1.46 (m, 2H), 1.51 (s, 3H) 1.06 (d, J=7.1 Hz, 3H), 0.85 (s, 3H), 0.85 (t, J=7.5 Hz, 3H). HRMS-ESI (m/z) calcd for C₃₇H₄₇NO₁₃Na [M+Na]⁺ 736.2940, found 736.2942.

Compound 27: MS-ESI (m/z) calcd for C₄₀H₅₄NO₁₃[M+H]⁺ 756.3595, found 756.3.

Compound 29: HRMS-ESI (m/z) calcd for C₃₆H₅₄NO₁₃ [M+H]⁺ 708.3590, found 708.3594.

Compound 30: MS-ESI (m/z) calcd for C₃₈H₅₈NO₁₃[M+H]⁺ 736.3980, found 736.4.

Compound 31: HRMS-ESI (m/z) calcd for C₃₃H₄₈NO₁₃ [M+H]⁺ 666.3120, found 666.3117.

Compound 32: MS-ESI (m/z) calcd for C₃₅H₅₂NO₁₃[M+H]⁺ 694.3439, found 694.3.

Compound 33: ¹H NMR (600 MHz, acetone-d₆, −40° C.) δ 5.85 (dd, J=7.2, 1.4 Hz, 1H), 5.67 (d, J=7.3 Hz, 1H), 5.53 (d, J=10.3 Hz, 1H), 5.41-5.34 (m, 1H), 5.16 (d, J=2.3 Hz, 1H), 5.01 (ddd, J=12.5, 5.0, 3.6 Hz, 1H), 4.70 (dd, J=4.0, 2.1 Hz, 1H), 4.01 (dd, J=15.9, 7.2 Hz, 1H), 3.68 (br s, 2H), 3.36 (br s, 2H), 3.15 (dd, J=10.4, 5.1 Hz, 1H), 2.74-2.61 (m, 3H), 2.56-2.51 (m, 2H), 2.51-2.44 (m, 2H), 2.41 (s, 3H), 2.23 (s, 6H), 2.21-2.02 (m, 4H), 2.02-1.98 (m, 1H), 1.96 (s, 3H), 1.90-1.84 (m, 1H), 1.56-1.45 (m, 2H), 1.51 (s, 3H), 1.10 (d, J=7.1 Hz, 3H), 0.86 (s, 3H), 0.85 (t, J=7.4 Hz, 3H). HRMS-ESI (m/z) calcd for C₃₇H₅₄NO₁₄ [M+H]⁺ 736.3539, found 736.3545.

Compound 34: ¹H NMR (600 MHz, acetone-d₆, −40° C.) δ 5.85 (dd, J=7.3, 1.3 Hz, 1H), 5.67 (d, J=7.2 Hz, 1H), 5.53 (d, J=10.3 Hz, 1H), 5.37 (dt, J=7.4, 1.7 Hz, 1H), 5.16 (d, J=2.4 Hz, 1H), 5.00 (ddd, J=12.4, 5.0, 3.6 Hz, 1H), 4.70 (dd, J=4.0, 2.1 Hz, 1H), 4.02 (dd, J=15.9, 7.2 Hz, 1H), 3.14 (dd, J=10.4, 5.1 Hz, 1H), 2.77-2.70 (m, 2H), 2.69-2.59 (m, 3H), 2.57-2.42 (m, 4H), 2.41 (s, 3H), 2.23 (s, 6H), 2.21-2.06 (m, 3H), 2.13 (s, 3H), 2.03-1.89 (m, 5H), 1.95 (s, 3H), 1.89-1.82 (m, 1H), 1.56-1.46 (m, 2H), 1.51 (s, 3H), 1.10 (d, J=7.1 Hz, 3H), 0.86 (s, 3H), 0.85 (t, J=7.4 Hz, 3H). HRMS-ESI (m/z) calcd for C₃₈H₅₇N₂O₁₃ [M+H]⁺ 749.3855, found 749.3864.

Compound 35: HRMS-ESI (m/z) calcd for C₃₆H₅₂NO₁₃ [M+H]⁺ 706.3433, found 706.3433.

Compound 36: ¹H NMR (600 MHz, acetone-d₆, −40° C.) δ 5.88-5.82 (m, 1H), 5.67 (d, J=7.3 Hz, 1H), 5.52 (d, J=10.4 Hz, 1H), 5.40-5.32 (m, 1H), 5.16 (d, J=2.3 Hz, 1H), 4.98 (dt, J=12.4, 4.3 Hz, 1H), 4.70 (dd, J=4.0, 2.1 Hz, 1H), 4.01 (dd, J=15.8, 7.2 Hz, 1H), 3.15 (dd, J=10.4, 5.1 Hz, 1H), 2.69-2.57 (m, 1H), 2.41 (s, 3H), 2.30 (td, J=7.6, 4.7 Hz, 2H), 2.23 (s, 3H), 2.22 (s, 3H), 2.20-1.99 (m, 4H), 1.95 (s, 3H), 1.89-1.82 (m, 1H), 1.73-1.57 (m, 6H), 1.57-1.45 (m, 2H), 1.50 (s, 3H), 1.45-1.37 (m, 2H), 1.21-1.06 (m, 3H), 1.09 (d, J=7.1 Hz, 3H), 0.90-0.77 (m, 2H), 0.86 (s, 3H), 0.85 (t, J=7.5 Hz, 3H). HRMS-ESI (m/z) calcd for C₃₉H₅₆O₁₃Na [M+H]⁺ 755.3613, found 755.3621.

Compound 42: ¹H NMR (600 MHz, acetone-d₆, −40° C.) δ 5.87 (dd, J=7.6, 2.6 Hz, 1H), 5.68 (d, J=7.3 Hz, 1H), 5.54 (d, J=10.3 Hz, 1H), 5.42-5.35 (m, 1H), 5.18 (d, J=2.4 Hz, 1H), 5.04 (dt, J=12.5, 4.3 Hz, 1H), 4.72 (dd, J=4.0, 2.1 Hz, 1H), 4.03 (dd, J=15.8, 7.3 Hz, 1H), 3.29 (d, J=2.5 Hz, 2H), 3.26, 3.20 (ABq, J=17.0 Hz, 2H), 3.18 (dd, J=17.2, 4.9 Hz, 1H), 2.99-2.93 (m, 1H), 2.86-2.75 (m, 2H), 2.73-2.60 (m, 3H), 2.42 (s, 3H), 2.37-2.27 (m, 4H), 2.24 (s, 6H), 2.22-2.08 (m, 4H), 1.97 (s, 3H), 1.92-1.86 (m, 1H), 1.58-1.47 (m, 2H), 1.52 (s, 3H), 1.11 (d, J=7.1 Hz, 3H), 0.87 (s, 3H), 0.87 (t, J=7.4 Hz, 3H). HRMS-ESI (m/z) calcd for C₃₉H₅₅N₂O₁₃ [M+H]⁺ 759.3699, found 759.3701.

Compound 44: HRMS-ESI (m/z) calcd for C₃₈H₅₆NO₁₃ [M+H]⁺ 734.3746, found 734.3741.

Compound 45: H NMR (600 MHz, acetone-d₆, −40° C.) δ 5.88-5.80 (m, 1H), 5.67 (d, J=7.3 Hz, 1H), 5.53 (d, J=10.3 Hz, 1H), 5.41-5.33 (m, 1H), 5.16 (d, J=2.4 Hz, 1H), 5.00 (ddd, J=12.5, 5.1, 3.7 Hz, 1H), 4.75-4.65 (m, 1H), 4.01 (dd, J=15.8, 7.2 Hz, 1H), 3.15 (dd, J=10.4, 5.1 Hz, 1H), 2.72 (br s, 2H), 2.66 (td, J=7.0, 5.2 Hz, 1H), 2.57 (br s, 4H), 2.44 (br s, 2H), 2.41 (s, 3H), 2.23 (s, 3H), 2.22 (s, 3H), 2.21-2.02 (m, 4H), 1.95 (s, 3H), 1.92-1.84 (m, 1H), 1.61-1.45 (m, 10H), 1.50 (s, 3H), 1.11 (d, J=7.1 Hz, 3H), 0.86 (s, 3H), 0.85 (t, J=7.3 Hz, 3H). HRMS-ESI (m/z) calcd for C₃₉H₅₈NO₁₃ [M+H]⁺ 748.3903, found 748.3900.

Compound 47: HRMS-ESI (m/z) calcd for C₃₉H₅₈NO₁₃ [M+H]⁺ 748.3903, found 748.3897.

Compound 48: HRMS-ESI (m/z) calcd for C₄₀H₆₀NO₁₃ [M+H]⁺ 762.4059, found 762.4049.

Compound 52: MS-ESI (m/z) calcd for C₃₈H₅₄NO₁₄ [M+H]⁺ 748.3544, found 748.3.

Compound 55: MS-ESI (m/z) calcd for C₃₉H₅₇N₂O₁₄ [M+H]⁺, 777.3810 found 777.4.

Compound 64: MS-ESI (m/z) calcd for C₃₈H₅₆NO₁₃ [M+H]⁺ 734.3752, found 734.4.

Compound 65: MS-ESI (m/z) calculated for C₃₉H₅₇N₂O₁₅ [M+H]⁺ 793.3759, found 793.4.

Compound 66: MS-ESI (m/z) calcd for C₄₁H₅₇N₂O₁₄ [M+H]⁺ 801.3810, found 801.4.

Compound 68: MS-ESI (m/z) calcd for C₄₁H₅₇N₃O₁₄ [M+H]⁺ 816.3919, found 816.4.

### Procedure D

Compounds of Formula (I-C1) can be prepared following the procedure illustrated in Scheme VI below.

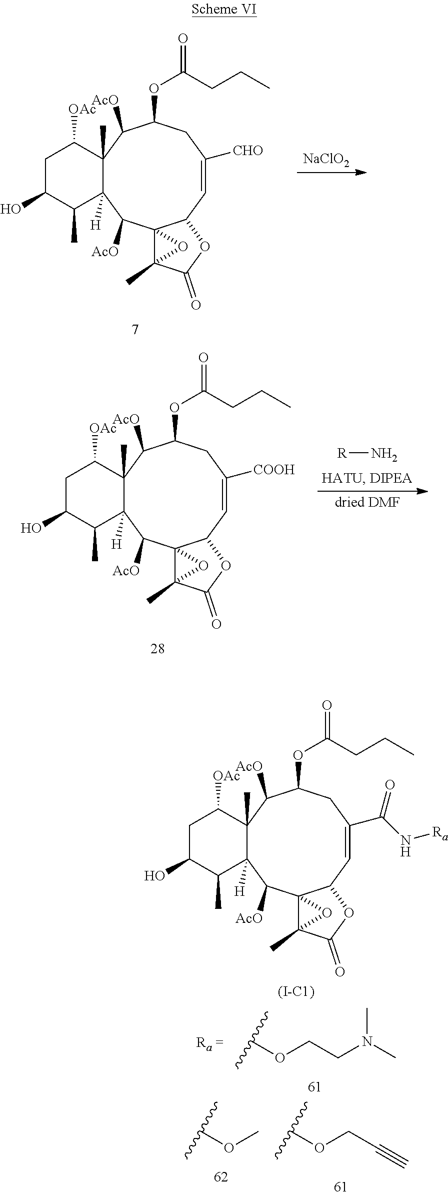

Compound 7 (100 mg, 0.164 mmol) is dissolved in t-butanol (0.8 mL), THF (0.8 mL), and 2-methyl-2-butene (0.3 mL, 3.28 mmol). A mixture of NaClO₂ (81.5 mg, 0.902 mmol) and NaH₂PO₄ (127.7 mg, 1.07 mmol) in water (0.3 mL) is then added to the Compound 7 solution. After being stirred at room temperature for 20 hrs, the reaction mixture is quenched with 1 N HCl (2 mL). Ethyl acetate is added. The organic layer is collected. The aqueous layer is extracted with ethyl acetate. The organic layers are combined, dried over anhydrous MgSO₄, filtered, and concentrated to obtain a residue, which is purified by flash column chromatography to afford Compound 28.

At room temperature, Compound 28 (1 equiv.) is mixed with amines (1.1 equiv., MilliporeSigma), 1-[Bis(dimethylamino)methylene]-1H-1,2,3-triazolo[4,5-b]pyridinium 3-oxid hexafluorophosphate (“HATU”, 1.5 equiv., MilliporeSigma), diisopropylethylamine (3 equiv.) and dried DMF (0.5 mL) or dried dichloromethane (0.5 mL, MilliporeSigma). The resultant mixture is stirred at room temperature for 20 hours, diluted with ethyl acetate, and then washed with water. The organic layer is collected and dried over anhydrous MgSO₄, filtered, and concentrated to obtain a residue, which is purified by flash column chromatography to afford a compound of Formula (I-C1).

The following compounds were prepared using Procedure D: Compounds 28 and 61-63.

Compound 28: HRMS-ESI (m/z) calcd for C₃₀H₄₀O₁₄Na [M+Na]⁺ 647.2310, found 647.2311.

Compound 61: MS-ESI (m/z) calcd for C₃₄H₅₁N₂O₁₄ [M+H]⁺ 711.3340, found 711.4.

Compound 62: MS-ESI (m/z) calcd for C₃₁H₄₄NO₁₄ [M+H]⁺ 654.2762, found 654.3.

Compound 63: MS-ESI (m/z) calcd for C₃₃H₄₄NO₁₄ [M+H]⁺ 678.2762, found 678.3.

### Procedure E

Compounds of Formula (I-A1) can be prepared following the procedure illustrated in Scheme VII below.

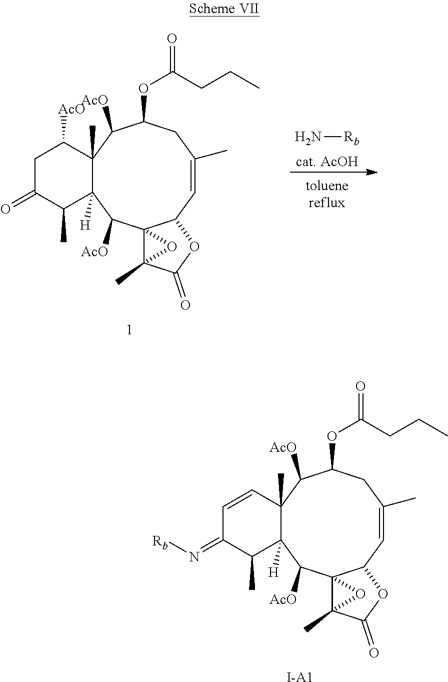

At room temperature, Compound 1 (1 molar equiv.) is mixed with hydrazine (1.5 equiv., MilliporeSigma), AcOH (2 drops) and toluene (0.3 mL). The resultant mixture is stirred at reflux for 20 hours, diluted with ethyl acetate, and then washed with water. The organic layer is collected and dried over anhydrous MgSO₄, filtered, and concentrated to obtain a residue, which is purified by flash column chromatography to afford a compound of Formula (I-A1).

The following compounds were prepared using Procedure E: Compounds 9-12 and 18-20.

Compound 9: MS-ESI (m/z) calcd for C₃₂H₄₅O₂₀ [M+H]⁺ 617.3407, found 617.3.

Compound 10: MS-ESI (m/z) calcd for C₃₃H₄₈O₃₉ [M+H]⁺ 630.3391, found 630.3.

Compound 11: MS-ESI (m/z) calcd for C₃₂H₄₃O₂₀ [M+H]⁺ 615.2918, found 615.3.

Compound 12: MS-ESI (m/z) calcd for C₃₂H₄₃O₂₀ [M+H]⁺ 615.2918, found 615.3.

Compound 18: MS-ESI (m/z) calcd for C₃₀H₄₁O₂₀ [M+H]⁺ 589.2761, found 589.3.

Compound 19: MS-ESI (m/z) calcd for C₃₀H₄₁O₂₀ [M+H]⁺ 589.2761, found 589.3.

Compound 20: MS-ESI (m/z) calcd for C₃₅H₄₃O₂₀ [M+H]⁺ 651.2918, found 651.3.

### Procedure F

Scheme VIII below depicts exemplary preparation of compounds of formula (I-H) of this invention.

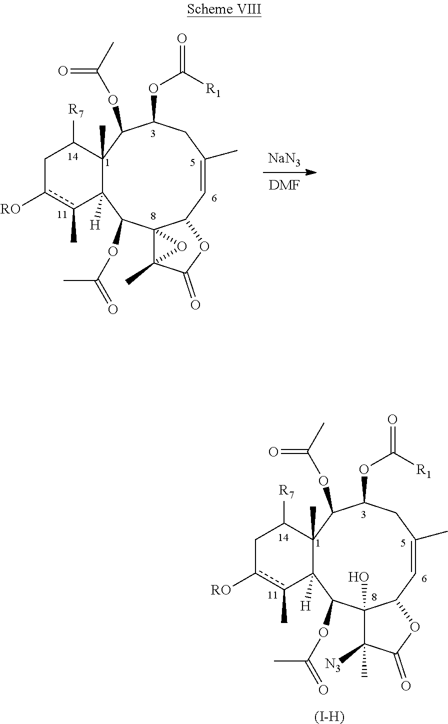

At room temperature, SP001 or SP040 (1 molar equiv.) is mixed with sodium azide (4 equiv., MilliporeSigma), DMF (0.3 mL). The resultant mixture is stirred at 80° C. for 16 hours, quenched with water, and then extracted with ethyl acetate. The combined organic layers were dried over anhydrous MgSO₄, filtered, and concentrated to obtain a residue, which is purified by flash column chromatography to afford a compound of Formula.

The following compounds were prepared using Procedure F: Compounds 2, 38 and 43.

Compound 2: HRMS-ESI (m/z) calculated for C₃₀H₄₁N₃O₁₁Na [M+Na]⁺ 642.2633, found 642.2633.

Compound 38: HRMS-FAB (m/z) calculated for C₃₀H₄₄N₃O₁₂ [M+H]⁺ 638.2925, found 638.2921.

Compound 43: MS-ESI (m/z) calculated for C₄₀H₅₅N₄O₁₃ [M+H]⁺ 799.3766, found 799.4.

### Procedure G

Scheme IX below illustrates a method for preparing compounds of formula (I-I) of this invention.

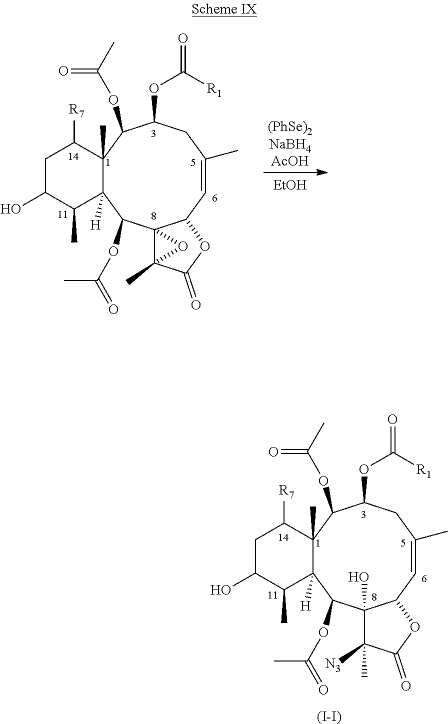

To a stirring solution of diphenyl diselenide (1.5 equiv.) in EtOH (0.6 mL) under a nitrogen atmosphere was added NaBH₄ (3 equiv.) at room temperature. After evolution of hydrogen ceased, the light-yellow solution was cooled to 0° C., followed by the addition of acetic acid (1.5 equiv.), and the mixture was further stirred at 0° C. for 50 min. The resulting colorless solution was then added to a solution of SP001 (1 equiv.) in EtOH (0.6 mL) at room temperature. The reaction mixture was diluted with ethyl acetate, and oxygen was passed through the solution for 5 minutes. The reaction mixture was washed with H₂O. The organic layer was collected, dried over MgSO₄, filtered, and concentrated by rotary evaporation. The residue was purified by flash column chromatography to afford a compound of Formula.

The following compounds were prepared using Procedure G: Compound 54.

Compound 54: HRMS-ESI (m/z) calculated for C₃₀H₄₄NO₁₂Na [M+Na]⁺ 619.2725, found 619.2735.

### Procedure H

Scheme X below shows an exemplary method of preparing compound 3 and its analogs of this invention.

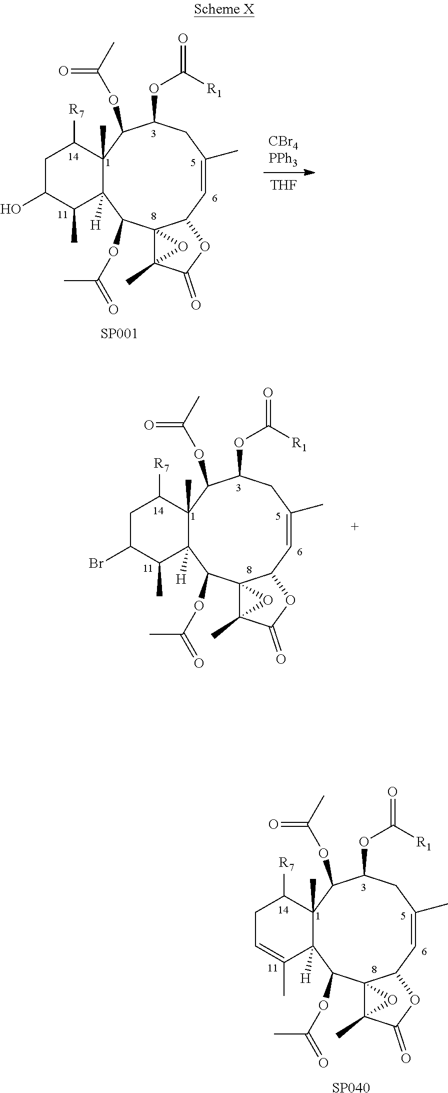

At room temperature, SP001 (1 equiv.) is mixed with CBr₄ (3.0 equiv., MilliporeSigma), PPh₃ (3.0 equiv., MilliporeSigma) and dried THF (4 mL). The resultant mixture is stirred at room temperature for 16 hours, the reaction mixture was filtered through a short pad of Celite to remove triphenylphosphine oxide and rinsed with ether. The filtrate was concentrated and purified by prep HPLC (C18) to afford a compound of Formula.

Compound 3 was prepared using Procedure H: HRMS-FAB (m/z) calculated for C₃₀H₄₂⁷⁹BrO₁₁ [M+H]⁺ 657.1910, found 657.1911; C₃₀H₄₂⁸¹BrO₁₁ [M+H]⁺ 659.1890, found 659.1880.

SP040 was prepared using Procedure H: HRMS-ESI (m/z) calculated for C₃₀H₄₀O₁₁Na [M+Na]⁺ 599.2463, found 599.2465.

### Procedure I

Scheme XI below illustrates a method of preparing compounds of formula (I-J) of this invention.

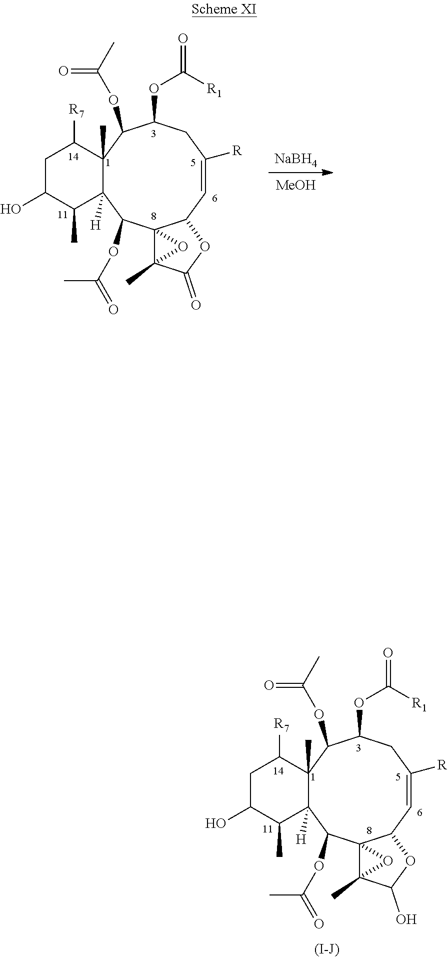

At room temperature, SP001 or compound 7 (1 molar equiv.) is mixed with sodium borohydride (2 equiv., MilliporeSigma), and methanol (0.3 mL). The resultant mixture is stirred at 0° C. for 3 hours, quenched with water, and then extracted with ethyl acetate. The combined organic layers were dried over anhydrous MgSO₄, filtered, and concentrated to obtain a residue, which is purified by flash column chromatography to afford a compound of Formula.

The following compounds were prepared using Procedure I: Compounds 39 and 46.

Compound 39: MS-ESI (m/z) calculated for C₃₀H₄₄NaO₁₃ [M+Na]⁺ 635.2680, found 635.3.

Compound 46: HRMS-ESI (m/z) calculated for C₃₀H₄₄O₁₂Na [M+Na]⁺ 619.2725, found 619.2734.

### Procedure J

Scheme XII below illustrates preparation of compounds of formula (I-G) of this invention.

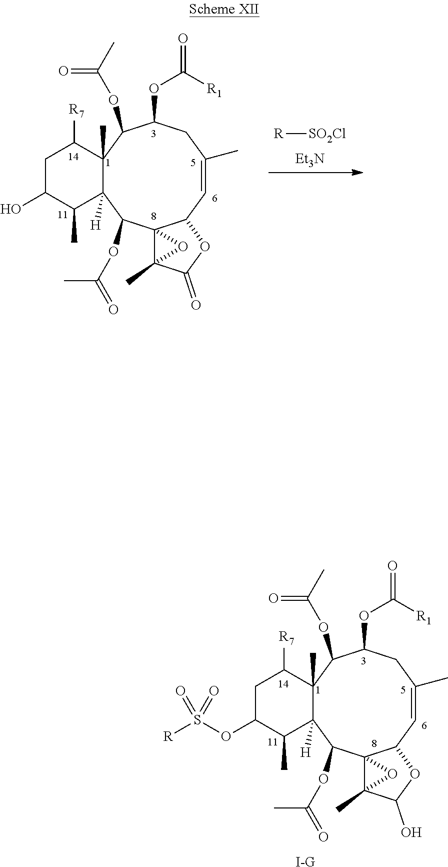

At room temperature, SP001 is mixed with RSO₂Cl (2 equiv.), triethylamine (3 equiv., MilliporeSigma) and dried DCM (0.4 mL). The resultant mixture is stirred at room temperature for 16 hours, quenched with water, and then extracted with DCM. The combined organic layers were dried over anhydrous MgSO₄, filtered, and concentrated to obtain a residue, which is purified by flash column chromatography to afford a compound of Formula.

The following compounds were prepared using Procedure J: Compounds 13, 14 and 49.

Compound 13: MS-ESI (m/z) calculated for C₃₀H₄₃NO₂₀SNa[M+Na]⁺ 696.2302, found 696.2.

Compound 14: MS-ESI (m/z) calculated for C₃₁H₄₅O₁₄S[M+H]⁺ 673.2530, found 673.2.

Compound 49: ¹H NMR (600 MHz, acetone-d₆, −40° C.) δ 5.80 (dd, J=7.3, 1.7 Hz, 1H), 5.66 (d, J=7.2 Hz, 1H), 5.55 (d, J=10.2 Hz, 1H), 5.32 (d, J=7.3 Hz, 1H), 5.16 (d, J=2.3 Hz, 1H), 4.94 (dt, J=12.6, 4.1 Hz, 1H), 4.76-4.72 (m, 1H), 4.00 (dd, J=15.9, 7.0 Hz, 1H), 3.45-3.33 (m, 2H), 3.18 (dd, J=10.2, 5.1 Hz, 1H), 2.96-2.76 (m, 3H), 2.65 (hept, J=7.1, 6.4 Hz, 2H), 2.41 (s, 3H), 2.34-2.28 (m, 1H), 2.25 (s, 3H), 2.23 (s, 3H), 2.22-2.06 (m, 4H), 1.97-1.84 (m, 2H), 1.91 (s, 3H), 1.61-1.39 (m, 11H), 1.15 (d, J=7.1 Hz, 3H), 0.87 (s, 3H), 0.85 (t, J=7.4 Hz, 3H). HRMS-ESI (m/z) calculated for C₃₇H₅₆NO₁₄S [M+H]⁺ 770.3416, found 770.3410.

### Procedure K

Scheme XIII below shows a method of preparing Compound 6 and its analogs of this invention.

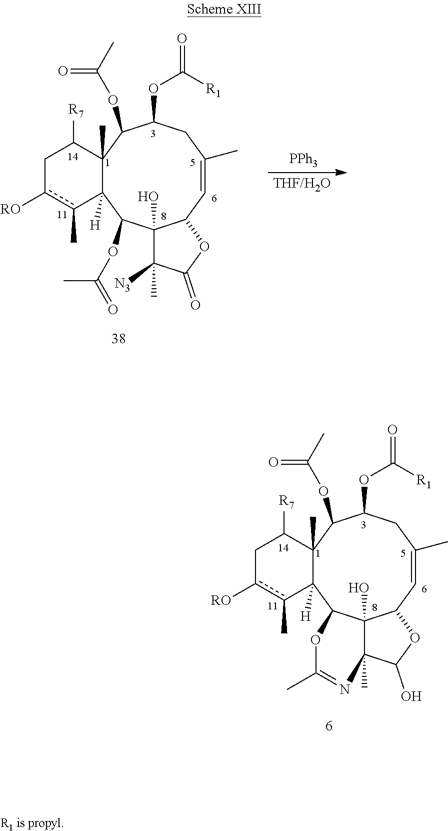

At room temperature, Compound 38 (1 equiv.) was mixed with PPh₃ (4 equiv.) and THF/H₂O (40:1, 1.2 mL). The resultant mixture was stirred at room temperature for 16 hours, quenched with water, and then extracted with ethyl acetate. The combined organic layers were dried over anhydrous MgSO₄, filtered, and concentrated to obtain a residue, which was purified by flash column chromatography to afford Compound 6.

MS-ESI (m/z) calculated for C₃₀H₄₂NO₁₀[M+H]⁺ 576.2809, found 576.3.

### Procedure L

Scheme XIV below shows a method of preparing Compound 50 and it analogs of this invention.

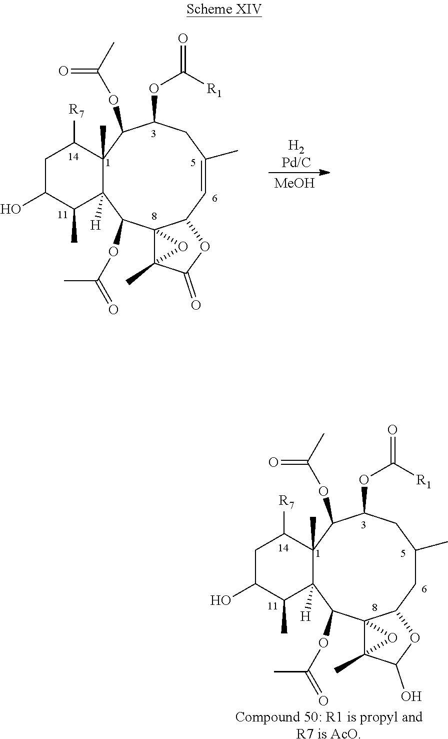

At room temperature, SP001 (1 equiv.) was mixed with Pd/C and methanol (1.5 mL). The resultant mixture was stirred under H₂ (balloon) at room temperature for 16 hours, filtered through a pad of Celite, and then rinsed with ethyl acetate. The filtrate was concentrated to obtain a residue, which was purified by flash column chromatography to afford compound 50: MS-ESI (m/z) calculated for C₃₀H₄₅O₁₂ [M+H]⁺ 597.2911, found 597.3.

### Procedure M

Compound 53 was prepared following the method shown in Scheme XV below.

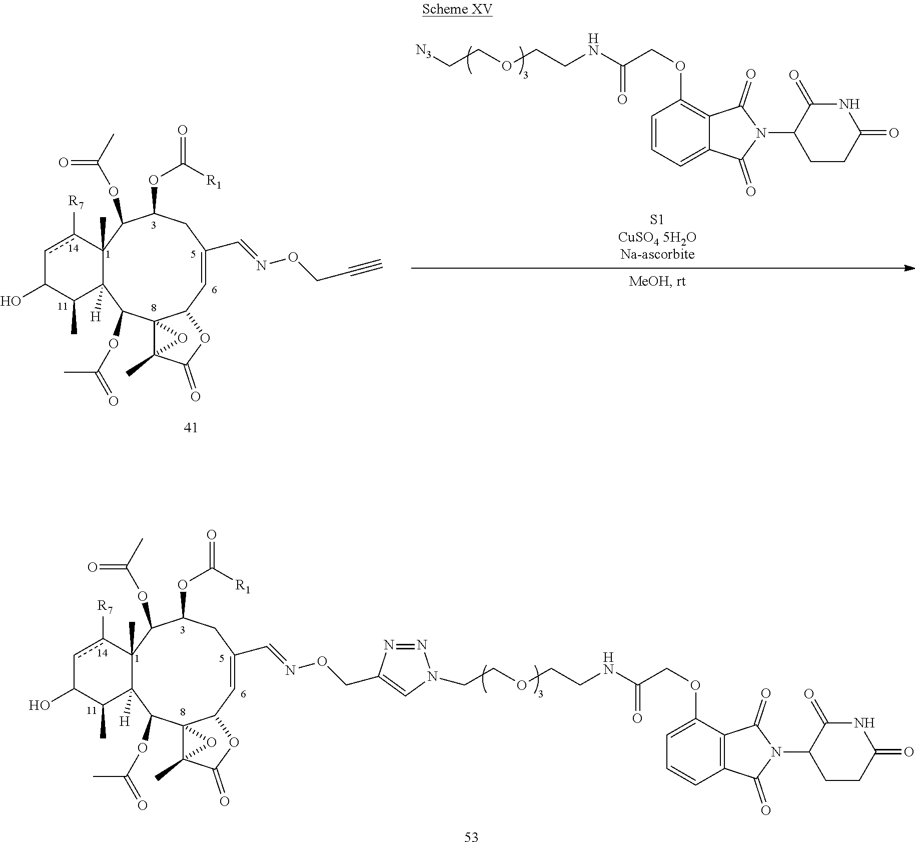

At room temperature, compound 41 (1 equiv.) was mixed with S1 (1 equiv.), CuSO₄ 5H₂O (0.2 equiv.), sodium ascorbate (2 equiv.) and MeOH (0.6 mL). The resultant mixture was stirred at room temperature for 20 min, filtered through a short pad of Celite and rinsed with ethyl acetate. The filtrate was collected and concentrated to obtain a residue, which was purified by flash column chromatography to afford compound 53.

MS-ESI (m/z) calculated for C₅₆H₇₂N₇O₂₂ [M+H]⁺ 1194.4730, found 1194.5.

Compound 69 was prepared following the method shown in Scheme XVI below.

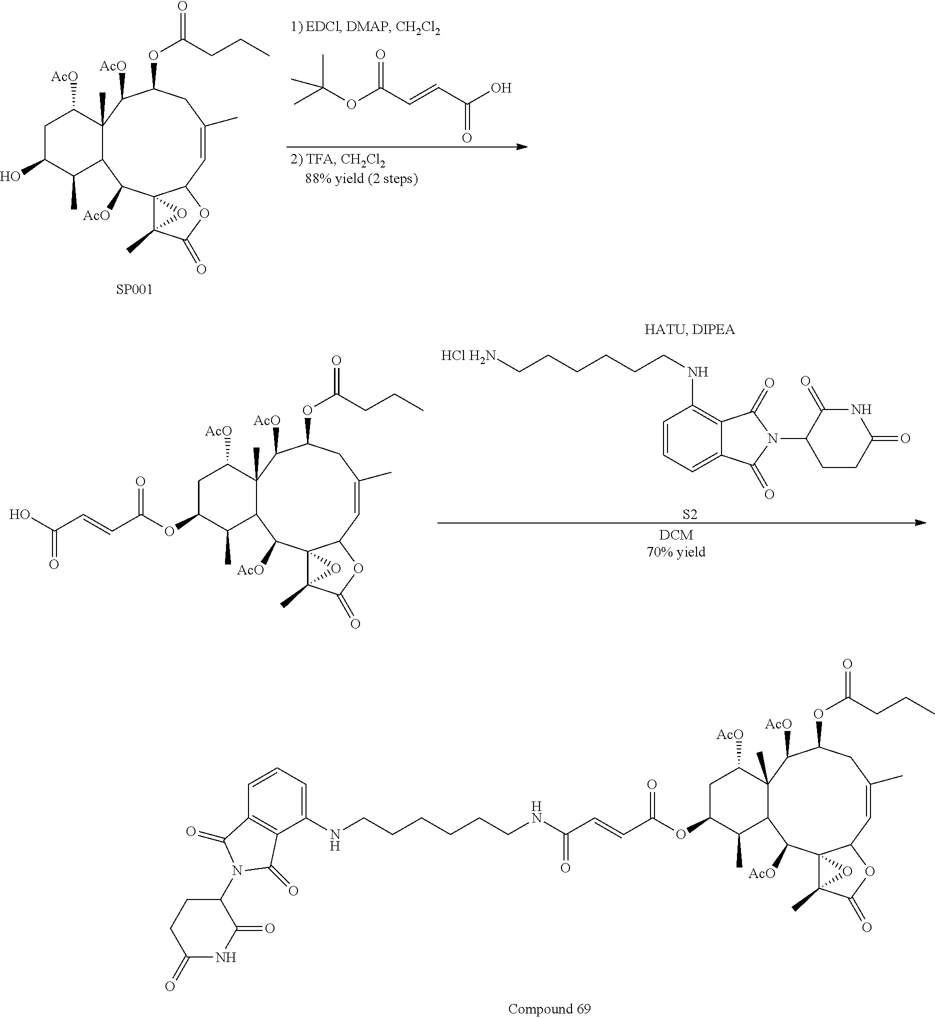

At room temperature, Compound SP001 (1 equiv.) was mixed with (E)-4-(tert-butoxy)-4-oxobut-2-enoic acid (1.2 equiv.), dimethylamino-pyridine (“DMAP”, 0.3 equiv., commercially available from MilliporeSigma, St. Louis, MO), 1-ethyl-3-(3-dimethylaminopropyl)carbodi-imide (“EDCI”, 3 equiv., MilliporeSigma) and dried CH₂Cl₂ (0.1 M). The resultant mixture was stirred for 18 hours, diluted with CH₂Cl₂, and then washed with water. The organic layer was collected and dried over anhydrous MgSO₄, filtered, and concentrated to obtain a residue, which was purified by flash column chromatography to afford an ester compound as a colorless oil used in the next step directly as an intermediate.

At room temperature, the above intermediate (1 equiv.) was mixed with TFA (2.5 equiv.) and CH₂Cl₂ (0.2 M). The resultant mixture was stirred at room temperature for 16 hours and then concentrated under vacuo. The residue was purified by flash column chromatography to afford a carboxylic compound.

At room temperature, the above carboxylic compound (1 equiv.) was mixed with HATU (1.2 equiv.), DIPEA (4 equiv.) and compound S2 (1 equiv.). The reaction mixture was stirred at room temperature for 18 hours, diluted with CH₂Cl₂, and then washed with water. The organic layer was collected and dried over anhydrous MgSO₄, filtered, and concentrated to obtain a residue, which was purified by prep HPLC (C18) to afford a compound 69: MS-ESI (m/z) calcd for C₅₃H₆₇N₄O₁₈ [M+H]⁺ 1047.4450, found 1047.4.

Compounds 70, 71, and 72 were prepared following the method shown in Scheme XVII below.

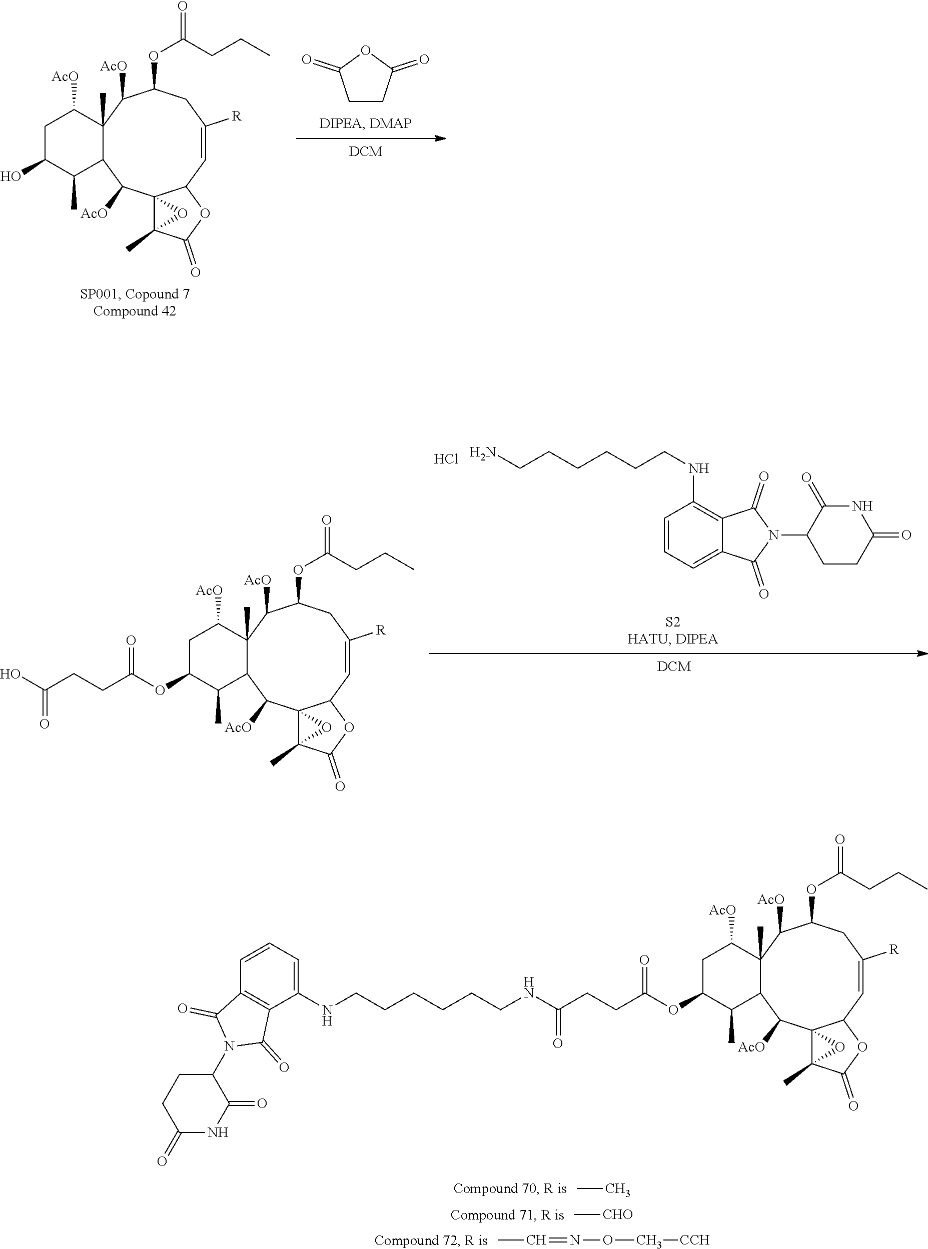

At room temperature, Compound SP001, Compound 7, or Compound 42 (1 equiv.) was mixed with succinic anhydride (3 equiv.), dimethylamino-pyridine (“DMAP”, 1.3 equiv., MilliporeSigma), DIPEA (3 equiv.) and dried CH₂Cl₂ (0.1 M). The resultant mixture was stirred at 40° C. for 18 hours, diluted with CH₂Cl₂, and then acidified with 1N HCl (aq.). The aqueous layer was extracted with CH₂Cl₂ for three times. The organic layers were collected and dried over anhydrous MgSO₄, filtered, and concentrated to obtain a residue, which was purified by flash column chromatography to afford a carboxylic intermediate as a colorless oil.

At room temperature, the above intermediate (1 equiv.) was mixed with HATU (1.2 equiv.), DIPEA (4 equiv.) and compound S2 (1 equiv.). The reaction mixture was stirred at room temperature for 18 hours, diluted with CH₂Cl₂, and then washed with water. The organic layer was collected and dried over anhydrous MgSO₄, filtered, and concentrated to obtain a residue, which is purified by prep HPLC (C18) to afford one of Compounds 70, 71 and 72.

Compound 70: MS-ESI (m/z) calcd for C₅₃H₆₉N₄O₁₈ [M+H]⁺ 1049.4607, found 1049.5.

Compound 71: MS-ESI (m/z) calcd for C₅₃H₆₇N₄O₁₉ [M+H] 1063.4400, found 1063.4.

Compound 72: MS-ESI (m/z) calcd for C₅₆H₇₀N₅O₁₉ [M+H] 1116.4665, found 1117.5.

The compounds thus prepared can be further purified following conventional methods such as crystallization, distillation/vacuum distillation, flash chromatography over silica, and preparative liquid chromatography.

Efficacy of the compounds of this invention can be initially determined using an in vitro method to identify their anti-inflammatory activity, all described in examples below. The selected compounds can be further tested to verify their in vivo efficacy, pharmacokinetic profiles, and toxicity, e.g., by administering it to an animal. Based on the results, an appropriate dosage range and administration route can be determined.

A compound of this invention is preferably formulated into a pharmaceutical composition containing a pharmaceutical carrier. The pharmaceutical composition is then given to a subject in need thereof to inhibit inflammation thus treating disorders associated therewith.

Without further elaboration, it is believed that one skilled in the art can, based on the above description, utilize the present invention to its fullest extent. The following examples are to be construed as merely illustrative and not limitative of the remainder of the disclosure in any way whatsoever.

All publications cited herein are hereby incorporated by reference in their entirety.

Set forth below are examples illustrating preparation and efficacy evaluation of compounds of this invention.

### Real-Time PCR Analysis for iNOS mRNA

Real time quantitative polymerase chain reaction (qPCR) was performed according to methods described by Livak et al. and De Gois et al. with modifications. See Livak et al., Methods 2001, 25, 402-408; and De Gois et al., J. Neurosci. 2005, 25, 7121-33.

Cell pellets were collected from centrifuged tubes after 8 hours. Total RNA was isolated using TRIzol® RNA Isolation Reagents (Life Technologies, Carlsbad, California) according to the manufacturer's instructions. After centrifugation at 3000 rpm for 8 minutes at 4° C., total RNA was obtained and transcribed using the iScript™ cDNA synthesis kit (Bio-Rad, Hercules, California). Reactions were performed in duplicate with 0.5 μL of each primer (0.2 μM final concentration), 25 μL of iQ SYBR Green Supermix™ (Bio-Rad, 100 mM KCl, 40 mM Tris-HCl, pH 8.4, 0.4 mM of each dNTP, iTaq DNA polymerase, 50 units/mL, 6 mM MgCl2, SYBR Green™ I, 20 nM fluorescein and stabilizer) and 2.5 μL of template in a 50-μL total volume. The PCR cycle conditions were 95° C. for 10 minutes, 40 cycle of 95° C. for 15 seconds, and then 60° C. for 1 minute. A melting curve analysis was performed at the end of each experiment to verify that a single product per primer pair was amplified. The amplification and analysis were performed using a CFX96 Touch™ Real-time PCR Detection System (Bio-Rad). Results were compared using the relative cycle threshold (CT) method. The fold increase or decrease was determined relative to a blank control after normalizing to a housekeeping gene (GAPDH) using 2-ΔΔCT. The real-time PCR oligonucleotide primers used for genotyping are as follows: iNOS (forward), 5′-GCTGTTAGAGACACTTCTGAG-3′; iNOS (reverse), 5′-CACTTTGGTAGGATTTGACTTTG-3′.

The inhibition of iNOS (%) was calculated as percent reduction of iNOS gene expression to that of LPS-stimulated iNOS gene expression as 100%. A high iNOS inhibition indicates that the compound can exert a reduction of LPS-stimulated iNOS gene expression greater than 80%.

Compounds 1-67 were evaluated by the assay described above. Each compound showed inhibition of iNOS gene expression up to 97%. Among them, Compounds 24, 34, 37, 41, 55, and 66 were very potent, having an inhibition of iNOS gene expression between 50% and 97%.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. For example, compounds structurally analogous to the compounds of this invention also can be made, screened for their efficacy in treating a condition that relates to SOS1. Thus, other embodiments are also within the claims.

What is claimed is:

1. A compound of Formula (I): 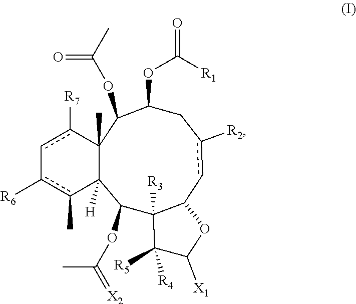
in which
R₁ is C₁-C₆ alkyl;
R₂ is C₁-C₆ alkyl, formyl, carboxyl, —CH═N—ORₐ, —CH═NRₐ, or —C(O)NH—O—Rₐ, in which Rₐ is H, C₁-C₆ alkyl, C₂-C₆ alkynyl, or C₁-C₆ heterocycloalkyl;
R₃ is H, hydroxy, or combined with R₄, and R₄ is H or C₁-C₆ alkyl; when R₃ and R₄ are combined, they together with the two carbon atoms attached thereto form an epoxide ring;
R₅ is N₃, methyl, or combined with X₂, and when R₅ and X₂ are combined, they are N and, together with the carbon atoms attached thereto, form a heterocycloalkyl ring;
R₆ is H, halo, oxo, hydroxyl, ═NR_{b}, ═N—NH—C(O)R_{c}, —OC(O)R_{d}, or —O—S(O)₂Rₑ, in which R_{b} is hydroxyl, C₁-C₆ alkoxy, or C₁-C₁₀ heterocycloalkyl, R_{c} is C₁-C₆ alkyl, C₃-C₆ cycloalkyl, 5 or 6-membered heterocycloalkyl, phenyl, or 5- or 6-membered heteroaryl, R_{d} is C₃-C₆ cycloalkyl, C₂-C₁₀ alkenyl, C₁-C₁₀ aminoalkyl, C₃-C₁₀ alkynylalkyl, C₁-C₁₀ heterocyclylalkyl, or phenyl, and Rₑ is amino or C₁-C₆ alkyl;
R₇ is H or acetyloxy;
X₁ is hydroxyl or oxo;
X₂ is O or N, when X₂ is N, it is combined with R₅;
each of [symbol] is a single bond or a double bond;
alkyl is unsubstituted or substituted with one or more groups selected from hydroxyl, amino, CN, aryl, heteroaryl, C₂-C₆ alkenyl, C₂-C₆ alkynyl, C₁-C₆ carboxylate, and C₁-C₆ heterocycloalkyl;
each of amino, aminoalkyl, alkenyl, alkoxy, cycloalkyl, alkynylalkyl, heterocyclylalkyl, heterocycloalkyl, heteroaryl, and phenyl, is unsubstituted or substituted with one or more groups selected from hydroxyl, C₁-C₆ alkyl, C₂-C₆ alkenyl, C₂-C₆ alkynyl, C₁-C₆ carboxylate, C₁-C₆ heterocycloalkyl, aryl, heteroaryl, and aryl-amino-alkyl-aminocarbonyl.

2. The compound of claim 1, wherein R₂ is formyl, carboxyl, —CH═N—ORₐ, —CH═NRₐ, or —C(O)NH—O—Rₐ; and R₆ is OH.

3. The compound of claim 1, wherein each of X₁ and R₆ is OH.

4. The compound of claim 1, wherein R₆ is oxo, ═NR_{b}, ═N—NH—C(O)R_{c}, —OC(O)R_{d}, or —O—S(O)₂Rₑ.

5. The compound of claim 1, wherein R₁ is methyl, ethyl, n-propyl, or iso-propyl.

6. The compound of claim 1, wherein each of R₃ and R₄, independently, is H or OH, or R₃ and R₄ are combined and, together with the carbon atoms they bond to, form an epoxide ring.

7. The compound of claim 1, wherein R₅ is H, OH, N₃, or methyl.

8. The compound of claim 1, wherein R₇ is acetyloxy.

9. The compound of claim 1, wherein the compound is one of Formulas (I-A), (I-B), (I-C), (I-D), (I-E), (I-F), and (I-G):

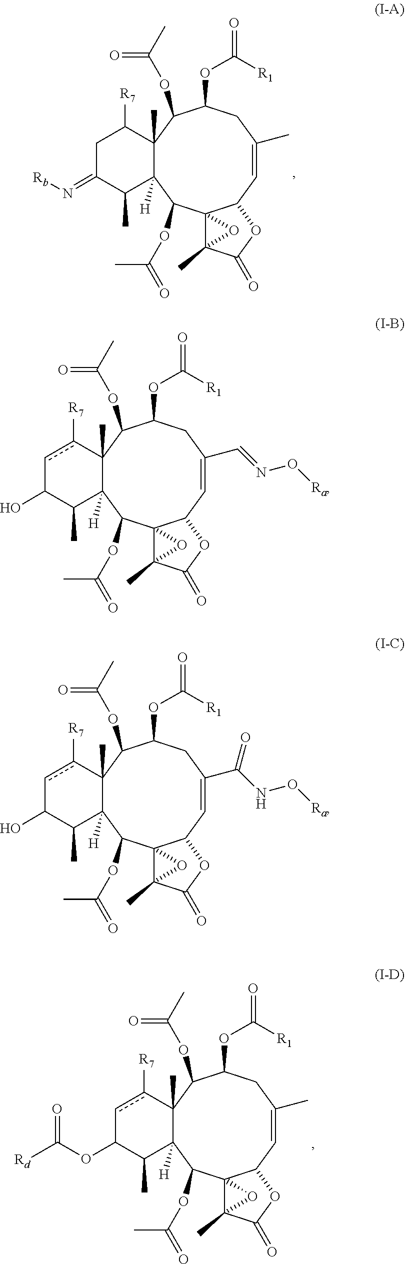

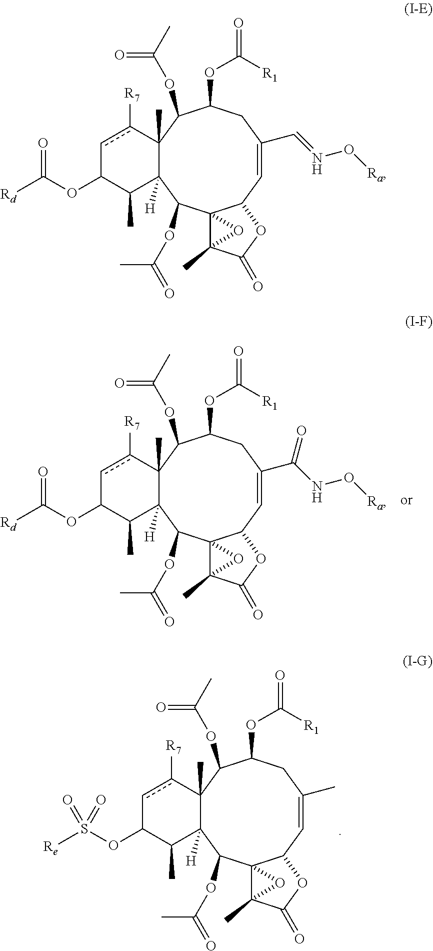

10. The compound of claim 9, wherein the compound is of Formula (I-A), and R_{b} is hydroxyl, C₁-C₆ alkoxy, 5- or 6-membered heterocycloalkyl, or —NH—C(O)R_{c}, R_{c} being C₁-C₆ alkyl, C₃-C₆ cycloalkyl, phenyl, or heteroaryl.

11. The compound of claim 10, wherein R₁ is propyl, R₇ is acetyloxy, and R_{b} is hydroxyl, methoxy, CH≡CCH₂O—, PhC(O)NH—, CH₃C(O)NH—, cyclopropanecarbox-amido, N-methylpiperazin-1-yl, or morpholino.

12. The compound of claim 9, wherein the compound is of Formula (I-B) or (I-C), wherein Rₐ is H, methyl, ethyl, prop-2-yn-1-yl, prop-2-en-1-yl, morpholino, benzyl, —CH₂CN, —CH₂CH₂N(CH₃)₂, or

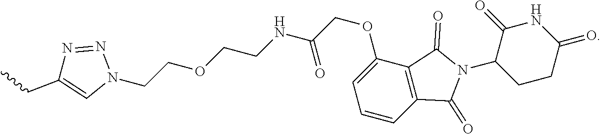

13. The compound of claim 9, wherein R₁ is propyl and R₇ is acetyloxy.

14. The compound of claim 9, wherein the compound is of Formula (I-B).

15. The compound of claim 9, wherein the compound if of Formula (I-C).

16. The compound of claim 9, wherein the compound is of Formula (I-D), (I-E), (I-F), or (I-G), wherein each of R_{d} and Rₑ, independently, is methyl, but-3-yn-1-yl, t-butyl-OC(O)—NHCH₂—, CH₃)₂NCH₂—, —NH₂, —CH₂NH₂, —CH₂CH₂NH₂, —CH₂CH₂N(CH₃)₂, —CH₂NHC(O)CH₃, —CH₂(CH₂)₄NH₂, —CH₂(CH₂)₄N(CH₃)₂,

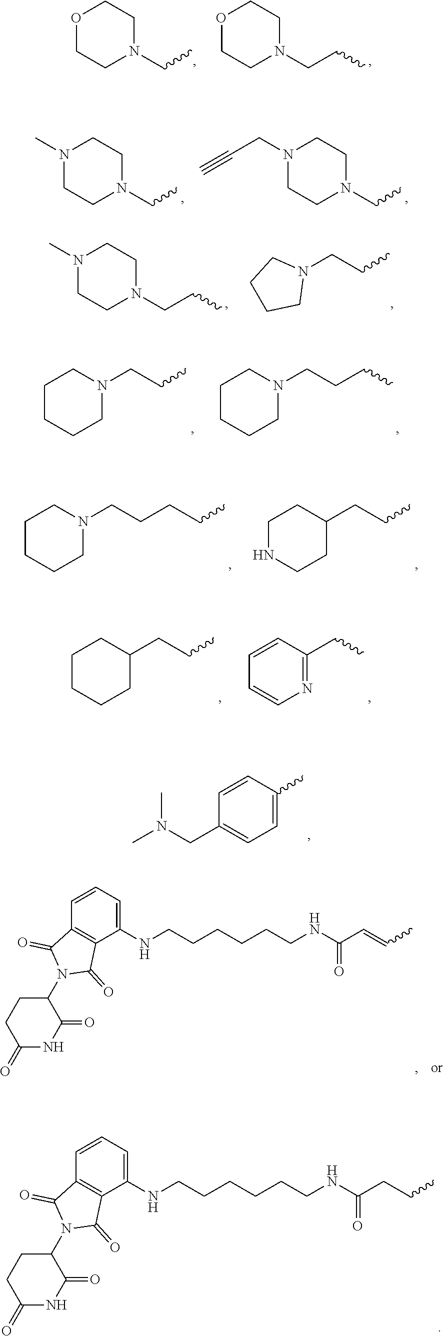

17. The compound of claim 16, wherein R₁ is propyl and R₇ is acetyloxy.

18. The compound of claim 9, wherein the compound is of Formula (I-E) or (I-F), and Rₐ is H, methyl, ethyl, prop-2-yn-1-yl, prop-2-en-1-yl, morpholino, benzyl, —CH₂CN, —CH₂CH₂N(CH₃)₂, or

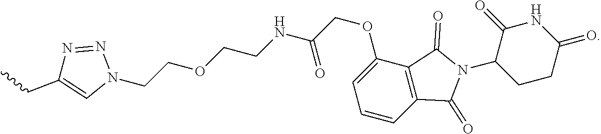

19. The compound of claim 1, wherein the compound is selected from Compounds 1-72.

20. A method of treating an inflammatory condition comprising administering to a subject in need thereof an effective amount of a compound of claim 1.

21. A pharmaceutical composition comprising a compound of claim 1 and a pharmaceutically acceptable carrier.


SEQUENCE LISTING (2 sequences)

SEQ ID NO: 1 - DNA, 21 bp, synthetic construct
  source 1..21: mol_type = other DNA
  misc_feature 1..21: note = iNOS forward primer
gctgttagagacacttctgag

SEQ ID NO: 2 - DNA, 23 bp, synthetic construct
  source 1..23: mol_type = other DNA
  misc_feature 1..23: note = iNOS reverse primer
cactttggtaggatttgactttg